(19) United States
(12) Patent Application Publication
(10) Pub. No.: US 2008/0242650 A1
(43) Pub. Date: Oct. 2, 2008

(54) Oral contraceptive regimen

(76) Inventor: Jean-Louis Thomas, Charenton-le-Pont (FR)

Correspondence Address: COOPER & DUNHAM, LLP, 1185 AVENUE OF THE AMERICAS, NEW YORK, NY, 10036, US

(21) Appl. No.: 12/079,335

(22) Filed: Mar. 25, 2008

Related U.S. Application Data
(60) Provisional application No. 60/920,326, filed on Mar. 26, 2007.

(51) Int. Cl. A61K 31/57; A61P 15/16

(52) U.S. Cl. 514/171

(57) ABSTRACT

A monophasic method of achieving contraception in a human female comprising orally administering to the human female a composition comprising 1.5 mg of 17-beta-estradiol and 2.5 mg of nomegestrol acetate for 24 days followed by a hormone-free period of 4 days.

Throughout this application, various publications are referenced in parentheses by author name and date. Full citations for these publications may be found at the end of the specification immediately preceding the claims. The disclosures of these publications in their entireties are hereby incorporated by reference into this application in order to more fully describe the state of the art as known to those skilled therein. However, the citation of a reference herein should not be construed as an acknowledgement that such reference is prior art to the present invention.

### BACKGROUND OF THE INVENTION

Most oral contraceptives (OCs) in use today are a combination of a synthetic estrogen, ethinylestradiol (EE), and a synthetic progestin, typically a 19-nortestosterone derivative. The monophasic OCs usually contain a fixed dose of EE and progestin to be taken for 21 days followed by 7 days without treatment. The period without treatment can be either a pill-free week or a one-week period of daily placebo tablet intake. In these OCs, the combination of the progestogen and the estrogen is responsible for the inhibition of ovulation. In addition to contributing to ovulation inhibition, EE is included in the composition to compensate for the reduced endogenous estrogenicity caused by the (effective) inhibition of ovarian function.

To decrease the risk of cardiovascular and thromboembolic events, the amount of EE has been progressively decreased and most preparations now contain 20 to 35 μg. In addition to contributing to ovulation inhibition, the progestin component induces changes in the cervical mucus (which hamper sperm transport) and the endometrium (which hamper implantation of the embryo).

Notwithstanding the foregoing there is still a desire to improve such OC products.

In order to do so, many attempts were made to replace ethinyloestradiol (EE) with the hormone naturally secreted by the ovaries, 17beta-oestradiol (E2), but none resulted in a product made available to women. In general, the anti-ovulatory effect was clearly obtained, but many of the failures were due to poor control of the desired cyclic vaginal bleeding profile, resulting in the appearance of intermenstrual spotting and bleeding which made the method unacceptable.

Thus, combinations of natural oestrogens with desogestrel (Wenzl, 1993; Kivinen and Saure, 1996; Csemicsky et al., 1996), with cyproterone acetate (Hirvonen et al., 1988; Hirvonen et al., 1995), with norethisterone (Astedt et al., 1977; World Health Organization, 1980; Serup et al., 1981) were found to be contraceptive, but the intermenstrual bleeding, spotting and poor quality of the periods were considered unacceptable. In some cases, the reason for these failures lay in an insufficient oestrogenic stimulation on account of the poor bioavailability of oestradiol or esters thereof; and an excessively intense progestative effect which led to a partial inhibition of endometrial proliferation and thus to anarchic bleeding (Hirvonen et al., 1995; Csemicsky et al., 1996). Only one combination gave satisfactory results in terms of controlling the menstrual cycle; a multiphasic combination of oestradiol valerate and dienogest (Oettel et al., 1999; Hoffman et al., 1999). According to these authors, the positive results were due to a strong dissociation between central activity (anti-ovulatory activity) and peripheral activity (endometrial activity) to the benefit of this latter activity for dienogest. Thus, previously published data show that the result depends closely on the anti-gonadotropic effect of the progestative agent, the bioavailability of oestradiol or derivatives thereof in the formulation used, an optimum ratio between the oestrogenic and progestative stimulations, and the specific regimen performed.

Attempts to manufacture a contraceptive combination drug product containing E2 have led to an OC which contains nomegestrol acetate (NOMAC) and estradiol (E2). Said oral contraceptive is disclosed in U.S. Pat. No. 6,906,049, in which the E2 1.5 mg/2.5 mg NOMAC is specifically disclosed. In this combination product, the contraceptive efficacy is mainly attributable to the progestin, a 19-norprogesterone derivative with a high gonadotropin-inhibiting effect (Bazin et al., 1987; Couzinet et al., 1999). Nomegestrol acetate is a powerful, orally-active progestative agent which has a novel pharmacological profile. Like 19-nor-testosterone derivatives, nomegestrol acetate possesses high anti-gonadotropic activity but, unlike these 19-nor-testosterone derivatives, it does not display any residual androgenic or oestrogenic activity and it has a strong anti-oestrogen activity. Like 17 alpha-hydroxyprogesterone derivatives, it has a pure pharmacological profile, but, unlike the above derivatives, it has a powerful anti-gonadotropic effect. It belongs to the category of progestative agents known as hybrids (Oettel et al., 1999) which do not display deleterious metabolic effects because of the absence of the 17 alpha-ethinyl function and combine the advantages of progesterone derivatives with those of the more modern 19-nor-testosterone derivatives. E2 is added to make the product acceptable in terms of cycle control, to compensate for the estrogen deficiency due to the inhibition of follicular growth by the progestin, and to reinforce the gonadotropin-inhibiting effect of NOMAC.

Generally, OCs are administered during 21 out of the 28 days of the woman cycle. However, some ovulations were observed with the above mentioned E2 1.5 mg/2.5 mg NOMAC 21-7 regimen. Some of them were associated with poor compliance, but they occurred in the first part of the cycle, which suggested excessive follicular growth during the drug-free interval.

It is known that during the drug-free interval, the absence of inhibitory steroids allows pituitary ovarian function to resume. There is a rise in FSH which elicits recruitment of follicles from which a dominant follicle can be selected. Comparing several low dose combination OCs, Van Heusden et al. concluded that the EE component rather than the progestin component determined the extent of residual ovarian activity during the drug-free interval (Van Heusden et al., 1999). They found that during this intercycle period the follicle diameters were statistically smaller in women treated with tablet containing 30 μg EE compared with two 20 μg EE tablets.

It was also shown that products containing 20 μg EE allow greater follicular development and higher E2 blood levels than those containing 30 μg of EE (Mall-Haefeli et al., 1991). Reducing the EE dose suggests that dose omission might lead more often to ovulation and contraceptive failure (Fitzgerald et al., 1994).

Reducing the drug-free interval to less than 7 days would be a means to decrease residual ovarian activity in women using low-dose combination OCs (Spona et al., 1996). Sullivan et al. compared the ovulation inhibition and the ovarian activity in women taking the same low-dose OCs containing 15 μg of EE and 60 μg of gestodene for either 21 or 24 days of each cycle (Sullivan et al., 1999). They demonstrated that reduction of the drug-free interval to 4 days was associated with more effective ovulation inhibition and less residual ovarian activity as compared to the conventional regimen with a 7-day drug-free interval. However, no significant difference was shown regarding the bleeding profile between the 21/7 and the 24/4 EE/gestodene regimens.

In the subject invention it has been found that the E2 1.5 mg/NOMAC 2.5 mg contraceptive combination administered monophasically for 24 out of 28 days provides a total duration of genital bleeding significantly shorter than did the 21/7 monophasic regimen. This shorter duration of genital bleeding is due to a shorter duration of both intermenstrual and withdrawal bleeding.

### SUMMARY OF THE INVENTION

The present invention provides a monophasic method of achieving contraception in a human female comprising orally administering to the human female a composition comprising 1.5 mg of 17-beta-estradiol and 2.5 mg of nomegestrol acetate for 24 days followed by a hormone-free period of 4 days.

The present invention also provides a monophasic method of achieving contraception in a human female wherein the duration of the genital bleeding is reduced. This method comprises orally administering to the human female a composition comprising 1.5 mg of 17-beta-estradiol and 2.5 mg of nomegestrol acetate for 24 days followed by a hormone-free period of 4 days.

This invention further provides a method of achieving contraception in a female human which comprises repeatedly performing the method described above e.g. performing the method again commencing on day 29.

### BRIEF DESCRIPTION OF THE FIGURES

The figures contain the following abbreviations: m (mean), SD (standard deviation), CI (confidence interval) and IU (International Unit).

FIG. 1. Mean diameter of the largest follicle with the 2 regimens in the ITT population (m±SD). The mean diameter of the largest follicle detected by vaginal ultrasound measurements at each assessment during the study for the 21-day and 24-day regimens in the intent to treat population.

FIG. 2. Mean diameter of the largest follicle with the 2 regimens in the PP population (m±SD). The mean diameter of the largest follicle detected by vaginal ultrasound measurements at each assessment during the study for the 21-day and 24-day regimens in the per-protocol population.

FIG. 3. Mean progesterone blood levels (ng/ml) with the 2 regimens in the ITT population (m±95% CI). The mean progesterone blood levels detected by vaginal ultrasound measurements at each assessment during the study for the 21-day and 24-day regimens.

FIG. 4. Mean estradiol blood levels (pg/ml) with the 2 regimens in the ITT population (m±95% CI). The mean estradiol blood levels by vaginal ultrasound measurements at each assessment during the study for the 21-day and 24-day regimens.

FIG. 5. Mean follicle stimulating hormone (FSH) blood levels (mIU/ml) with the 2 regimens in the ITT population (m±95% CI). The mean FSH blood levels detected by vaginal ultrasound measurements at each assessment during the study for the 21-day and 24-day regimens.

FIG. 6. Mean luteinizing hormone (LH) blood levels (mIU/ml) with the 2 regimens in the ITT population (m±95% CI). The mean LH blood levels detected by vaginal ultrasound measurements at each assessment during the study for the 21-day and 24-day regimens.

FIG. 7. Individual values of the follicular diameter ≧13 mm in the ITT population. The individual values of the follicular diameter for women with a follicle more than 13 mm in diameter during treatment in each regimen group.

FIG. 8. Diameter of the largest follicle in non-treatment compliant women. The diameter of the largest follicle measured for three non-compliant women in each group during the corresponding non-compliant cycle.

FIG. 9. Subject Disposition. Flow chart demonstrating the disposition of subjects through completion of the study.

### DETAILED DESCRIPTION OF THE INVENTION

“Return to fertility” means the presence of progesterone levels in blood of >3 ng/ml, measured around day 20 (and a few days Later, if necessary) and spontaneous menstruation occurring after the end of treatment.

“Withdrawal bleeding” means the occurrence of scheduled bleeding as related to the pill-free period or period of daily intake of placebo tablets.

“Breakthrough bleeding/spotting” (also named intermenstrual bleeding) means irregular or unscheduled bleeding, i.e., bleeding while taking active pills, i.e. any occurrence of vaginal bleeding outside the withdrawal bleeding episodes

“Absence of withdrawal bleeding” means the absence of scheduled bleeding in the pill-free (or placebo pill) interval.

“Intermenstrual duration” means the interval, i.e., number of days between the first day of 2 consecutive withdrawal bleedings.

“Ovulation” shall mean the presence of a follicle that was >13 mm in diameter and ruptured within a few days combined with blood progesterone level >3 ng/ml.

“Compliant subject” means any subject compliant with the daily intake of tablets (active and/or placebo) and associated treatment regimen (21-7 versus 24-4) during all treatment cycles.

“Genital bleeding” during the treatment period means the spontaneous menstruation occurring at the end of the pre-treatment cycle, the withdrawal bleedings occurring after treatment cycles 1 and 2 and all intermenstrual bleeding recorded between these three bleeding episodes.

A “blister pack” is a package containing a single cycle of study medication, either 21 tablets of 1.5 mg E2 and 2.5 mg NOMAC plus 7 placebo tablets, or 24 tablets of 1.5 mg E2 and 2.5 mg NOMAC plus 4 placebo tablets, provided by the investigator to each subject at the start of treatment. Each blister pack bore a label with the following items: name and address of the sponsor, protocol number, cycle number, treatment duration, route of administration, names of ingredients, subject identification number, batch number, subject's initials and expiry date.

“Treatment cycle” consisted of 21- or 24-days of once-a-day treatment with E2 1.5 mg/NOMAC 2.5 mg followed by placebo for 7 or 4 days, respectively. Subjects were instructed to take the first pill of study medication on the first day, but no later than day 3 of their natural menstrual bleeding.

“Treatment compliant” means that, for any given cycle, no pill was missed from day 1 to day 24 (inclusive) or no more than one dose was missed in this period provided the subject took two doses the day after, and absence of NOMAC serum levels below the limit of quantification during the active treatment. Treatment compliance was determined from review of the diaries completed for each treatment cycle and by account of the number of pills of study medication in each cycle in the blister packs returned by subjects. Compliance with mention of all missed tablets was recorded in the case record form (CRF) by the investigator. NOMAC plasma levels were measured in all blood samples (except day 27) collected throughout the study.

An “assessment” means performance of a vaginal ultrasound and obtainment of a blood sample for the determination of pituitary and ovary hormone levels.

A “per-protocol cycle” means that during the active treatment period (21 or 24 days) the subjects missed no pill or no more than one dose, provided the subject took two doses after the missed dose; no NOMAC blood levels measured during the active treatment period were below the limit of quantification; no more than two consecutive endovaginal ultrasounds were missing.

A “per-protocol population” (PP population) includes all subjects who were treatment compliant and fulfilled the three per-protocol cycle conditions given above.

The “intent to treat” population (ITT population) includes all randomized subjects who started treatment and had at least one efficacy assessment (endovaginal ultrasound to measure the diameter of follicles) during any treatment cycle.

“Eligible subject” includes women who complied with the following criteria: gave written informed consent; between 18 and 38 years of age; in general good health; cooperative regarding compliance with trial requirements and correctly filling out the subject diary card; had intact uterus and ovaries; had stopped previous use of oral contraception, intra uterine devices (IUD's) or implants 2 months before study drug intake (i.e. Visit 1); a resident of the town or the nearby surroundings of the investigational site during the trial period; agreed to use of condoms during sexual intercourses luring the whole study; had a previous cycle of 28±7 days (i.e. Last cycle before Visit 1); blood sample results were considered as normal by the investigator; has a benign Pap smear within the Last 18 months; had a negative pregnancy test; had a progesterone blood level >3 ng/ml (9 nmol/l) during the pre-treatment cycle; had a subject body mass index (BMI) 17≦BMI≦30; In addition, to be considered an “eligible subject,” a woman could not have any one of the following criteria: unable to use oral contraceptive in the past; a history of allergy or intolerance to the study drug; pregnant or lactating; a history of, or current thrombo-embolic disease (arterial or venous); a history of, or current hypertension (diastolic blood pressure >90 mmHg measured on more than 3 consecutive occasions) or history of pre-eclamptic syndrome; a history of, or current cardiovascular disease: coronary artery disease, valvulopathy, thrombogenic cardiac rhythm disturbances, cerebrovascular disease or ocular disease of vascular origin; a history of, or current cancer; a history of, or current severe fibrocystic breast disease (such as Reclus's disease); a history of pituitary tumour; known renal insufficiency; a history of, or current severe respiratory insufficiency or asthma; severe and frequent headaches or migraines; epilepsy; a history of systemic lupus erythematosus or other connective tissue disorders; a history of porphyria; a history of otosclerosis; a history of sickle cell anaemia; a history of severe or recent liver disease; a history of recurrent or pregnancy-related cholestasis; known diabetes mellitus type I or II; an endocrine disease: hypo- or hyper-thyroidism, Cushing's syndrome or acromegaly; a history of, or current severe endometriosis; under forfeiture of freedom or under guardianship; smoked 10 or more cigarettes a day; currently treated with, or had taken within the last 2 months prior to inclusion (i.e. Visit 1) estroprogestin or progestin treatment; currently treated with, or had taken within the last 2 months prior to inclusion (i.e. Visit 1), enzyme inducers (rifampicin, barbiturates, hydantoin, primidone, carbamazepine or griseofulvin); currently participating in another clinical trial or to have taken part in a clinical trial within the month prior to selection (i.e. Visit 0); had on the pelvic ultrasound: a myoma bigger than 30 mm or an uterine submucosal myoma; had on the pelvic ultrasound an ovarian mass to be investigated; had a haemoglobin level <10 g/dl; or presented a positive laboratory test for Hepatitis B surface antigen (HbsAg), HIV 1 and 2 antibodies and HCV antibody.

This invention provides a method, i.e. a monophasic method, of achieving contraception in a human female comprising orally administering to the female human a composition comprising 1.5 mg of 17-beta-estradiol (E2) and 2.5 mg of nomegestrol acetate (NOMAC) for 24 days followed by a hormone-free period of 4 days.

This invention further provides a method of achieving contraception in a human female wherein the duration of the genital bleeding is reduced, comprising orally administering to the human female a composition comprising 1.5 mg of 17-beta-estradiol and 2.5 mg of nomegestrol acetate for 24 days followed by a hormone-free period of 4 days.

This invention also provides the method of achieving contraception recited herein, wherein the composition is in the form of a tablet, and such tablet contains conventional binders, excipients and the like.

This invention further provides a method of achieving contraception in a female human which comprises repeatedly performing the method recited herein, e.g. commencing the method again on day 29.

It is further envisaged that a placebo may be administered daily during the hormone-free period.

Experimental Details

The following Experimental Details are set forth to aid in an understanding of the invention and are not intended, and should not be construed, to limit in any way the invention as set forth in the claims which follow hereafter.

Introduction

This Regimen Validation Trial (RVS), a single center, double-blind, two parallel group randomized study, was designed to compare two regimens of the same contraceptive combination of E2 1.5 mg and NOMAC 2.5 mg given 21 and 24 out of 28 days for 3 consecutive cycles.

The primary objective of the study was to compare the effect on ovarian activity of the same combination (E2 1.5 mg/NOMAC 2.5 mg) given in two cyclical regimen: 21 out of 28 days (drug-free interval: 7 days) and 24 out of 28 days (drug-free interval: 4 days). Ovarian activity was evaluated by monitoring follicular maturation with endovaginal ultrasound repeatedly during a 3-cycle period with special focus during the drug-free intervals. Pituitary and ovarian hormones were measured in the same time.

The secondary objectives were to evaluate the effects of the E2/NOMAC combination on cervical mucus using the Insler score; to assess bleeding control; to determine incidence of ovulation and luteal unruptured follicle (LUF) syndrome; to confirm “return to fertility” during the post-treatment cycle; and to establish the hormonal profiles throughout the treatment period (FSH, LH, E2 and Progesterone).

Materials and Methods

Disposition of Subjects

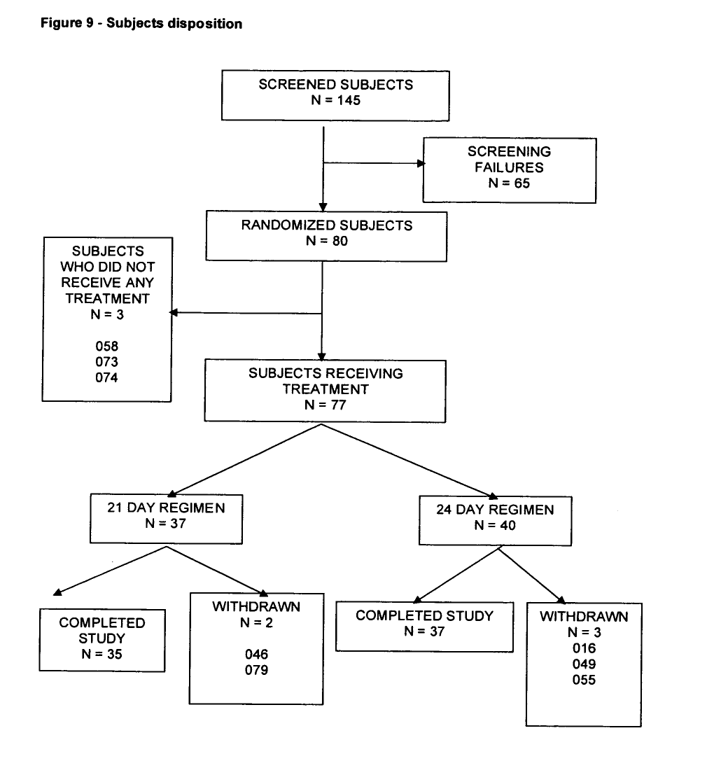

All the subjects were recruited in a single centre. One hundred and forty five premenopausal women (18 to 38 years old) were screened for this study, 80 were randomized. The main reason for which 65 subjects were excluded after screening was failure to meet inclusion criteria (29% of subjects screened did not meet the criteria: blood progesterone ≧3 ng/ml). Among the 80 randomized subjects, 3 of them withdrew their consent before taking any study treatment and were excluded from the analysis. Seventy seven subjects were treated: 37 in 21-day regimen group and 40 in 24-day regimen group. Of the 77 women who were randomized and treated, 5 (6.5%) did not complete the study. The reasons for withdrawal are given in Table 1. The disposition of subjects is illustrated in FIG. 9.

TABLE 1
Reasons of withdrawal

| | 21-day regimen | 24-day regimen |
|---|---|---|
| | (N = 37) | (N = 40) |
| Withdrawal of consent (%) | 0 | 2 (5.0) |
| Not compliant with the protocol (%) | 1 (2.7) | 1 (2.5) |
| Wrong inclusion (%) | 1 (2.7) | 0 |
| Total (%) | 2 (5.4) | 3 (7.5) |

The primary end-point used to calculate the sample size was the diameter of the largest follicle during the second and third treated cycles. On the basis of a previous study (Sullivan et al., 1999), the minimum expected difference between groups, considered as clinically significant, was 5 mm. The estimated standard deviation was 5.5 mm. The sample size required to detect this difference at the 0.05 level was 30 subjects per group. Assuming that 20% of subjects would drop out of the study or would not be evaluable, approximately 40 subjects per group were required to be included.

Pre-Treatment Cycle

Eligible subjects entered the pre-treatment cycle and were provided with diaries in which they were to record days on which genital bleeding or spotting occurred.

Women who used OCs, IUDs or contraceptive implants were to discontinue use of these methods two months before starting treatment and were offered barrier contraceptives to use during a pre-treatment menstrual cycle and throughout the treatment period.

Clinical evaluations, including measurement of weight, systolic and diastolic blood pressure, were performed before and after treatment and three times during the treatment period.

During the pre-treatment cycle, blood samples for the determination of pituitary and ovary hormones were to be obtained on approximately day 20. These samples were frozen and processed. Women who had a progesterone level ≧3 ng/ml were eligible to continue in the study. At the end of week 3 or 4 of the pre-treatment cycle, each subject was to have a vaginal ultrasound examination performed.

Near the end of the pre-treatment cycle, when the results of the progesterone assays and clinical chemistry and hematology were known, all subjects had a pregnancy test performed. Non pregnant women who met all study eligibility criteria were randomized to treatment for 3 cycles with the 21- or 24-day regimen. Forty subjects were to be randomly assigned to each regimen group.

Tablets Containing E2 1.5 mg/NOMAC 2.5 mg

The present study was designed to determine which of two different regimens produced the strongest follicular growth inhibition. The following drug supplies were used in the study for each treatment cycle: (i) 21 tablets of 1.5 mg E2 and 2.5 mg NOMAC plus 7 placebo tablets; (ii) 24 tablets of 1.5 mg E2 and 2.5 mg NOMAC plus 4 placebo tablets. Tablets containing E2 and NOMAC and placebo tablets were identical in appearance. The identical appearance of the two kinds of tablets was checked by a test of similarity before the beginning of the study. Each cycle of study medication was packaged in a blister pack. The blister packs were included in each subject kit that was

Labelled with the same information on each blister pack. Subjects were provided by the investigator a blister pack for each cycle at the start of treatment (blister pack 1), at the end of cycle 1 (blister pack 2), and during cycle 2 (blister pack 3). An additional blister pack was included in the subject kit, to be used if necessary (deterioration or loss of a blister pack by the subject). Subjects were randomly assigned to receive the E2/NOMAC combination either for 21 days followed by 7 placebo tablets or for 24 days followed by 4 placebo tablets. For each treatment cycle, subjects were to take one tablet each day from their blister pack. In treatment cycle 1, subjects were instructed to take the first tablet on the first day of menstrual bleeding or if not possible on days 2 or 3 of the cycle. Each dose of study medication was to be taken at the same time each day, at night. The 3 cycles of study medication were taken consecutively and continuously.

Data Recordation by Subjects During Treatment Cycles

For each treatment cycles, subjects were provided with diaries in which they were to record each day if they took study medication and days on which vaginal spotting or bleeding occurred. They had also to give during each treatment cycle what they considered to be the first day of withdrawal bleeding.

Clinical Assessments

At beginning of treatment, on about days 21, 24 and 27 of cycle 1 and days 2, 5, 8, 11, 13, 16, 21, 24 and 27 of cycles 2 and 3, subjects were to have vaginal ultrasound examinations performed and blood samples obtained for the determination of pituitary and ovary hormones levels. These assessments were repeated on about day 20 of the post-treatment cycle. Blood progesterone during the post-treatment cycle was also measured. Ultrasound examination and hormone measurements are standard and appropriate means of evaluating the efficacy of two regimens of the same contraceptive in the suppression of follicular maturation and ovulation (Van Heusden et al., 1999; Mall-Haefeli et al., 1991; Spona et al., 1996, Sullivan et al., 1999). All vaginal ultrasounds were performed in the same clinic by the same two operators with the same ultrasonograph (frequency 3 to 8.5 MHz).

Subjects were scheduled for clinic visits at inclusion, towards the end of treatment cycle 1 and on about day 13 of treatment cycles 2 and 3, and of post-treatment cycle. At these clinic visits, use of concomitant medications was evaluated, vital signs were taken, subjects were assessed for adverse events, the use of the diary cards was reviewed and completed diary cards collected. At clinic visit during treatment cycles 2 and 3 and post-treatment cycle, women had a breast and pelvic examination with assessment of cervical mucus and a pregnancy test. At the end of treatment cycle 3, blood samples were also obtained for clinical chemistry and haematology.

The schedule of assessments during the study is presented in Table 2.

TABLE 2
Schedule of Assessments

| | Pre-treatment | | Treated cycle n^{o}1 | | | | Treated cycle n^{o} 2 | | | | |
|---|---|---|---|---|---|---|---|---|---|---|---|
| | Days ± 1 | | | | | | | | | | |
| | D13 | D20 | INCL# | D21 | D24 | D27 | D2 | D5 | D8 | D11 | D13 |
| | Visits | | | | | | | | | | |
| | | | | | V2 | | | | | | V3 |
| | V0 | | V1 | | Follow | | | | | | Follow |
| | Screening | | Inclusion | | up | | | | | | up |
| Safety blood test | X | | | | | | | | | | |
| General physical | X | | | | | | | | | | |
| examination | | | | | | | | | | | |
| TA/Weight | X | | X | | X | | | | | | X |
| Pregnancy blood test | | X | | | | X | | | | | |
| Progesterone | | X | X | X | X | X | X | X | X | X | X |
| Estradiol/Estrone | | | | | | | | | | | |
| FSH/LH serum | | | | | | | | | | | |
| samples | | | | | | | | | | | |
| Gynaecological and | X | | | | | | | | | | X |
| breast examination | | | | | | | | | | | |
| insler score | | | | | | | | | | | |
| Pap smear | X^{⋄} | | | | | | | | | | |
| Endovaginal pelvic | | X* | X | X | X | X | X | X | X | X | X |
| ultrasound | | | | | | | | | | | |
| Blister-pack given | | | 1 | | 2 | | | | | | 3 |
| Diary card given | | | 1 | | 2 | | | | | | 3 |
| Diary card returned/ | | | | | | | | | | | 1 |
| Blister-pack returned | | | | | | | | | | | |

| | |
|---|---|
| Adverse Events | ←——————————————————————————→ |

| | | | | | | | | | | | | Post | |
|---|---|---|---|---|---|---|---|---|---|---|---|---|---|
| | Treated cycle n^{o} 2 | | | | Treated cycle n^{o} 3 | | | | | | | treatment | |
| | Days ± 1 | | | | | | | | | | | | |
| | D16 | D21 | D24 | D27 | D2 | D5 | D8 | D11 | D13 | D16 | D21 | D13 | D20 |
| | Visits | | | | | | | | | | | | |
| | | | | | | | | | V4 | | | V5 | |
| | | | | | | | | | Follow | | | Last | |
| | | | | | | | | | up | | | visit | |
| Safety blood test | | | | | | | | | | | X | | |
| General physical | | | | | | | | | X | | | X | |
| examination | | | | | | | | | | | | | |
| TA/Weight | | | | | | | | | X | | | X | |
| Pregnancy blood test | | | | X | | | | | | | X | | X |
| Progesterone | X | X | X | X | X | X | X | X | X | X | X | | X |
| Estradiol/Estrone | | | | | | | | | | | | | |
| FSH/LH serum | | | | | | | | | | | | | |
| samples | | | | | | | | | | | | | |
| Gynaecological and | | | | | | | | | X | | | X | |
| breast examination | | | | | | | | | | | | | |
| insler score | | | | | | | | | | | | | |
| Pap smear | | | | | | | | | | | | | |
| Endovaginal pelvic | X | X | X | X | X | X | X | X | X | X | X | | X* |
| ultrasound | | | | | | | | | | | | | |
| Blister-pack given | | | | | | | | | | | | | |
| Diary card given | | | | | | | | | | | | | |
| Diary card returned/ | | | | | | | | | 2 | | | 3 | |
| Blister-pack returned | | | | | | | | | | | | | |

| | |
|---|---|
| Adverse Events | ←—————————————————————————————————→ |

INCL #: 1^{st} day (2^{nd} day or 3^{rd} day) of menstrual bleedings
1: (V1) treatment and diary card for cycle 1 given to the subject
2: (V2) treatment and diary card for cycle 2 given to the subject
3: (V3) treatment and diary card for cycle 3 given to the subject
X*: Pelvic ultrasound including uterus measurements
X^{⋄}: Except if last pap smear < 18 months

Statistical Analysis of Data

All data manipulation, tabulation of descriptive statistics and statistical tests were performed using SAS version 8.2 for Windows system. All statistical tests of significance were performed as two-sided tests and a difference resulting in a p-value of ≦0.05 was considered statistically significant.

The analytical methods used for the statistical analysis are summarized in Table 3.

TABLE 3
Analytical Methods for Planned Analyses

| Statistical | | |
|---|---|---|
| Methods | Purpose | Variable analytical |
| Student t test | Baseline | Age, weight, BMI, systolic and |
| | Analysis | diastolic blood pressure, duration of |
| | | the last cycle, age at menarche, |
| | | diameter of the largest follicle and |
| | | hormonal concentrations. |
| | Efficacy | Mean diameter of the largest follicle, |
| | Analysis | mean hormonal concentrations, time to |
| | | onset of withdrawal bleeding, mean |
| | | duration of withdrawal and |
| | | intermenstrual bleeding at each cycle |
| | | and on all treated period, endometrial |
| | | thickness. |
| Wilcoxon Rank | Baseline | Number of pregnancies, number of |
| test | Analysis | childbirths, and Insler Score at |
| | | screening. |
| | Efficacy | Day of cycle corresponding to onset of |
| | Analysis | withdrawal bleeding and Insler Score at |
| | | cycle 2 and cycle 3. |
| Wilcoxon signed- | Efficacy | Change from baseline to cycle 2 and |
| Rank test | Analysis | cycle 3 in Insler Score |
| Chi-square test or | Baseline | Ethnic origin, smoking habits, overall |
| Fisher's exact | Analysis | results of physical and gynecological |
| test | | examination |
| | Efficacy | Number of subjects with follicle >10 mm, |
| | Analysis | with follicle >13 mm, with more |
| | | than one follicle >10 mm on the same |
| | | ultrasound. Number of subjects at each |
| | | cycle and number of cycles with |
| | | withdrawal bleeding, with at least one |
| | | day of intermenstrual bleeding. |
| | Safety | Incidence of adverse events, number of |
| | Analysis | subjects with at least one adverse |
| | | event. |
| ANOVA model | Safety | Change from baseline to cycle 3 in mean |
| with treatment | Analysis | systolic and diastolic blood pressure, |
| as factor and | | weight and standard laboratory tests at |
| baseline value as | | the end of Treatment cycle 3. |
| covariate | | |

Adverse events were coded using MedDRA dictionary before unblinding. MedDRA system organ classes (SOC) and preferred terms were used for the statistical summaries of adverse event data.

Results

Intent to Treat Population Demographic and Baseline Characteristics

Overall, the 76 subjects of the Intent to Treat population were 19-39 years of age (mean 27.4 years), 69.7% were Caucasian, 29.0% Black and 1.3% Asian. There was no significant difference across regimen groups concerning age and ethnic origin (Table 4).

TABLE 4
Demographic Characteristics, ITT Population

| | 21-day regimen | 24-day regimen | |
|---|---|---|---|
| Characteristics | (N = 37) | (N = 39) | P-value |
| Age (years) | | | |
| Mean ± SD | 26.3 ± 4.9 | 28.5 ± 4.8 | 0.053 |
| Range | 19-38 | 20-38 | |
| Race | | | |
| Caucasian n (%) | 23 (62.2) | 30 (76.9) | 0.130 |
| Black n (%) | 14 (37.8) | 8 (20.5) | |
| Asian n (%) | 0 | 1 (2.6) | |

There were no significant difference between regimen groups in the mean age at menarche, mean duration of last menstruation cycle, gravidity and parity, and use of tobacco (less than 10 cigarettes per day as required by the protocol) (Table 5). For all women, the mean age at menarche was 12.7 years (range 9-16 years), the mean duration of last menstrual cycle was 28.6 days (range: 25-32 days), 56.6% were nulligravid, 79.0% were nulliparous and 42.1% smoked.

TABLE 5
Gynecological History and Tobacco Use, ITT Population

| | 21-day regimen | 24-day regimen | |
|---|---|---|---|
| | (N = 37) | (N = 39) | P-value |
| Age at menarche (years) | | | |
| Mean ± SD | 12.7 ± 1.5 | 12.7 ± 1.4 | 0.974 |
| Range | 9-16 | 10-16 | |
| Duration of last menstrual | | | |
| cycle (days) | | | |
| Mean ± SD | 28.7 ± 1.6 | 28.4 ± 1.3 | 0.412 |
| Range | 25-32 | 25-32 | |
| Nulligravid n (%) | 22 (59.5) | 21 (53.9) | 0.622 |
| Nulliparous n (%) | 30 (81.1) | 30 (76.9) | 0.657 |
| Tobacco n (%) | 18 (48.7) | 14 (35.9) | 0.260 |

There were no remarkable differences across regimen groups in the proportions of subjects with medical histories and/or concomitant diseases and of subjects taking allowed concomitant therapy (Table 6 and Table 7).

TABLE 6
Medical history and/or concomitant diseases (ITT)

| | TOTAL | | 21 days | | 24 days | | |
|---|---|---|---|---|---|---|---|
| | N | % | N | % | N | % | P values |
| NO | 11 | 14.47 | 4 | 10.81 | 7 | 17.95 | 0.3767 |
| YES | 65 | 85.53 | 33 | 89.19 | 32 | 82.05 | |
| TOTAL | 76 | 100.00 | 37 | 100.00 | 39 | 100.00 | |

TABLE 7
Concomitant therapy (ITT)

| | TOTAL | | 21 days | | 24 days | | |
|---|---|---|---|---|---|---|---|
| | N | % | N | % | N | % | p Value |
| NO | 53 | 69.74 | 27 | 72.97 | 26 | 66.67 | 0.5497 |
| YES | 23 | 30.26 | 10 | 27.03 | 13 | 33.33 | |
| TOTAL | 76 | 100.00 | 37 | 100.00 | 39 | 100.00 | |

Subjects in the two regimen groups were not significantly different with respect to their mean weight, body mass index, or systolic and diastolic blood pressure (Table 8).

TABLE 8
Physical Examination, ITT Population

| | 21-day regimen | 24-day regimen | |
|---|---|---|---|
| | (N = 37) | (N = 39) | P-value |
| Weight (kg)) | | | |
| Mean ± SD | 60.8 ± 8.1 | 61.3 ± 8.5 | 0.777 |
| Range | 48-82 | 45-78 | |
| BMI (kg/m²) | | | |
| Mean ± SD | 22.4 ± 2.7 | 22.7 ± 3.1 | 0.726 |
| Range | 17-29 | 18-30 | |
| Systolic blood pressure | | | |
| (mmHg) | | | |
| Mean ± SD | 114.9 ± 10.2 | 114.8 ± 10.3 | 0.958 |
| Range | 94-137 | 101-145 | |
| Diastolic blood pressure | | | |
| (mmHg) | | | |
| Mean ± SD | 63.0 ± 6.7 | 62.4 ± 6.1 | 0.674 |
| Range | 46-77 | 52-79 | |

The gynecological examination, the characteristics of the cervical mucus evaluated with the Insler Score and the Pap smears were comparable across regimen groups (Table 9 to Table 11). There were only few abnormal findings at the gynecological examination and on Pap smears, which were not considered as clinically significant.

TABLE 9
Gynecological examination (ITT)

| | | TOTAL | | 21 days | | 24 days | | p |
|---|---|---|---|---|---|---|---|---|
| | | N | % | N | % | N | % | Values |
| VOLVA EXAMINATION | NORMAL | 76 | 100.00 | 37 | 100.00 | 39 | 100.00 | — |
| | TOTAL | 76 | 100.00 | 37 | 100.00 | 39 | 100.00 | |
| VAGINAL EXAMINATION | NORMAL | 75 | 98.68 | 36 | 97.30 | 39 | 100.00 | 0.4868 |
| | ABNORMAL NOT CS* | 1 | 1.32 | 1 | 2.70 | | | |
| | TOTAL | 76 | 100.00 | 37 | 100.00 | 39 | 100.00 | |
| CERVIX EXAMINATION | NORMAL | 74 | 97.37 | 36 | 97.30 | 38 | 97.44 | 1.0000 |
| | ABNORMAL NOT CS* | 2 | 2.63 | 1 | 2.70 | 1 | 2.56 | |
| | TOTAL | 76 | 100.00 | 37 | 100.00 | 39 | 100.00 | |
| UTERUS EXAMINATION | NORMAL | 76 | 100.00 | 37 | 100.00 | 39 | 100.00 | — |
| | TOTAL | 76 | 100.00 | 37 | 100.00 | 39 | 100.00 | |
| OVARY EXAMINATION | NORMAL | 76 | 100.00 | 37 | 100.00 | 39 | 100.00 | — |
| | TOTAL | 76 | 100.00 | 37 | 100.00 | 39 | 100.00 | |
| BREAST EXAMINATION | NORMAL | 75 | 98.68 | 36 | 97.30 | 39 | 100.00 | 0.4868 |
| | ABNORMAL NOT CS* | 1 | 1.32 | 1 | 2.70 | | | |
| | TOTAL | 76 | 100.00 | 37 | 100.00 | 39 | 100.00 | |

*CS: Clinically Significant

TABLE 10
Insler Score (ITT)

| | TOTAL | | 21 days | | 24 days | | |
|---|---|---|---|---|---|---|---|
| INSLER SCORE | N | % | N | % | N | % | p Value |
| 1 | 1 | 1.32 | | | 1 | 2.56 | 0.1183 |
| 2 | 5 | 6.58 | 2 | 5.41 | 3 | 7.69 | |
| 3 | 5 | 6.58 | 3 | 8.11 | 2 | 5.13 | |
| 4 | 4 | 5.26 | 3 | 8.11 | 1 | 2.56 | |
| 5 | 9 | 11.84 | 6 | 16.22 | 3 | 7.69 | |
| 6 | 12 | 15.79 | 8 | 21.62 | 4 | 10.26 | |
| 7 | 7 | 9.21 | 4 | 10.81 | 3 | 7.69 | |
| 8 | 6 | 7.89 | 1 | 2.70 | 5 | 12.82 | |
| 9 | 27 | 35.53 | 10 | 27.03 | 17 | 43.59 | |
| TOTAL | 76 | 100.00 | 37 | 100.00 | 39 | 100.00 | |

TABLE 11
Pap smear (ITT)

| | TOTAL | | 21 days | | 24 days | | |
|---|---|---|---|---|---|---|---|
| PAP SMEAR | N | % | N | % | N | % | p Value |
| NORMAL | 72 | 94.74 | 34 | 91.89 | 38 | 97.44 | 0.4800 |
| ABNORMAL NOT | 2 | 2.63 | 1 | 2.70 | 1 | 2.56 | |
| CS | | | | | | | |
| INADEQUACY | 2 | 2.63 | 2 | 5.41 | | | |
| TOTAL | 76 | 100.00 | 37 | 100.00 | 39 | 100.00 | |

Subjects in the two regimen groups were not significantly different with respect to findings at baseline endovaginal ultrasound. Overall there were 32.9 and 19.7% of women with at least one follicle more than 10 and 13 mm in diameter respectively. The mean diameter of the largest follicle was 8.8±5.14 mm (Table 12 and Table 13).

TABLE 12
Endovaginal ultrasound (ITT)

| | | | | | | | | | p |
|---|---|---|---|---|---|---|---|---|---|
| Variable | Regimen | N | MIN | MAX | MEAN | MEDIAN | SD | SEM | Values |
| UTERUS LENGTH | 21 days | 37 | 40 | 74 | 59.4 | 59.0 | 6.47 | 1.06 | 0.7262 |
| | 24 days | 39 | 46 | 78 | 59.9 | 59.0 | 7.63 | 1.22 | |
| | ALL | 76 | 40 | 78 | 59.6 | 59.0 | 7.05 | 0.81 | |
| UTERUS WIDTH | 21 days | 37 | 27 | 59 | 40.4 | 41.0 | 7.22 | 1.19 | 0.3352 |
| | 24 days | 39 | 27 | 66 | 42.1 | 41.0 | 8.42 | 1.35 | |
| | ALL | 76 | 27 | 66 | 41.3 | 41.0 | 7.86 | 0.90 | |
| UTERUS THICKNESS | 21 days | 37 | 24 | 44 | 33.4 | 34.0 | 5.08 | 0.84 | 0.6032 |
| | 24 days | 39 | 24 | 49 | 32.8 | 34.0 | 5.13 | 0.82 | |
| | ALL | 76 | 24 | 49 | 33.1 | 34.0 | 5.08 | 0.58 | |
| ENDOMETRIAL THICKNESS | 21 days | 37 | 4 | 14 | 7.9 | 8.0 | 2.21 | 0.36 | 0.2584 |
| | 24 days | 39 | 2 | 13 | 7.3 | 7.0 | 2.46 | 0.39 | |
| | ALL | 76 | 2 | 14 | 7.6 | 7.0 | 2.35 | 0.27 | |
| RIGHT OVARY DIAMETER | 21 days | 37 | 20 | 46 | 30.9 | 29.0 | 6.25 | 1.03 | 0.2288 |
| | 24 days | 39 | 17 | 41 | 29.2 | 29.0 | 5.67 | 0.91 | |
| | ALL | 76 | 17 | 46 | 30.0 | 29.0 | 5.98 | 0.69 | |
| LEFT OVARY DIAMETER | 21 days | 37 | 19 | 50 | 30.8 | 29.0 | 6.34 | 1.04 | 0.4396 |
| | 24 days | 39 | 20 | 41 | 29.7 | 29.0 | 5.29 | 0.85 | |
| | ALL | 76 | 19 | 50 | 30.2 | 29.0 | 5.81 | 0.67 | |

TABLE 13
Endovaginal ultrasound - Follicles (ITT)

| | TOTAL | | 21 days | | 24 days | | |
|---|---|---|---|---|---|---|---|
| PRESENCE OF FOLLICLE | N | % | N | % | N | % | p Value |
| NO | 5 | 6.58 | 3 | 8.11 | 2 | 5.13 | 0.6705 |
| YES | 71 | 93.42 | 34 | 91.89 | 37 | 94.87 | |
| TOTAL | 76 | 100.00 | 37 | 100.00 | 39 | 100.00 | |

| Variable | Regimen | N | MIN | MAX | MEAN | MEDIAN | SD | SEM | p Value |
|---|---|---|---|---|---|---|---|---|---|
| DIAMETER OF | 21 days | 37 | 0 | 31 | 9.8 | 9.0 | 5.99 | 0.98 | 0.1316 |
| THE LARGEST | 24 days | 39 | 0 | 18 | 8.0 | 7.0 | 4.07 | 0.65 | |
| FOLLICLE | ALL | 76 | 0 | 31 | 8.8 | 8.0 | 5.14 | 0.59 | |

| WOMEN WITH DIAMETER OF THE | TOTAL | | 21 days | | 24 days | | |
|---|---|---|---|---|---|---|---|
| LARGEST FOLLICLE > 10 mm | N | % | N | % | N | % | p Value |
| NO | 51 | 67.11 | 22 | 59.46 | 29 | 74.36 | 0.1670 |
| YES | 25 | 32.89 | 15 | 40.54 | 10 | 25.64 | |
| TOTAL | 76 | 100.00 | 37 | 100.00 | 39 | 100.00 | |

| NUMBER OF FOLLICLES WITH | TOTAL | | 21 days | | 24 days | |
|---|---|---|---|---|---|---|
| DIAMETER > 10 mm | N | % | N | % | N | % |
| 0 | 51 | 67.11 | 22 | 59.46 | 29 | 74.36 |
| 1 | 24 | 31.58 | 14 | 37.84 | 10 | 25.64 |
| 2 | 1 | 1.32 | 1 | 2.70 | | |
| TOTAL | 76 | 100.00 | 37 | 100.00 | 39 | 100.00 |

| WOMEN WITH DIAMETER OF THE | TOTAL | | 21 days | | 24 days | | p |
|---|---|---|---|---|---|---|---|
| LARGEST FOLLICLE > 13 mm | N | % | N | % | N | % | Value |
| NO | 61 | 80.26 | 27 | 72.97 | 34 | 87.18 | 0.1199 |
| YES | 15 | 19.74 | 10 | 27.03 | 5 | 12.82 | |
| TOTAL | 76 | 100.00 | 37 | 100.00 | 39 | 100.00 | |

At baseline, pituitary and ovary hormones (LH, FSH, estradiol and progesterone) and carrier proteins (SHBG, CBG and TBG), measured at Day 20 of the pre-treatment cycle were similar across regimen groups (Table 14 and Table 15). As requested by the protocol, all women had ovulation in the pre-treatment cycle, as assessed by a progesterone blood level ≧3 ng/ml (Table 15).

TABLE 14
Pituitary and ovary hormones and carrier proteins (ITT)

| | | | | | | | | | p |
|---|---|---|---|---|---|---|---|---|---|
| Variable | Regimen | N | MIN | MAX | MEAN | MEDIAN | SD | SEM | Values |
| FSH | 21 days | 36 | 1.7 | 15.1 | 3.93 | 3.07 | 2.655 | 0.442 | 0.3532 |
| | 24 days | 38 | 1.5 | 14.5 | 4.50 | 3.76 | 2.559 | 0.415 | |
| | ALL | 74 | 1.5 | 15.1 | 4.22 | 3.53 | 2.604 | 0.303 | |
| E2 | 21 days | 36 | 92.0 | 447.0 | 221.34 | 197.50 | 88.921 | 14.820 | 0.5253 |
| | 24 days | 38 | 51.4 | 522.0 | 207.91 | 186.00 | 91.954 | 14.917 | |
| | ALL | 74 | 51.4 | 522.0 | 214.44 | 196.00 | 90.124 | 10.477 | |
| P | 21 days | 36 | 0.1 | 22.0 | 10.08 | 8.94 | 7.024 | 1.171 | 0.8729 |
| | 24 days | 38 | 0.5 | 23.8 | 9.83 | 10.08 | 6.105 | 0.990 | |
| | ALL | 74 | 0.1 | 23.8 | 9.95 | 9.58 | 6.523 | 0.758 | |
| LH | 21 days | 36 | 0.2 | 78.8 | 6.70 | 3.11 | 13.123 | 2.187 | 0.7105 |
| | 24 days | 38 | 0.2 | 32.7 | 5.80 | 3.91 | 6.775 | 1.099 | |
| | ALL | 74 | 0.2 | 78.8 | 6.23 | 3.56 | 10.297 | 1.197 | |
| E1 | 21 days | 36 | 58.3 | 448.0 | 166.48 | 139.50 | 85.782 | 14.297 | 0.4986 |
| | 24 days | 38 | 76.1 | 325.0 | 153.93 | 133.50 | 72.718 | 11.796 | |
| | ALL | 74 | 58.3 | 448.0 | 160.03 | 137.00 | 79.045 | 9.189 | |
| SHBG | 21 days | 36 | 18.8 | 128.0 | 64.69 | 63.05 | 21.706 | 3.618 | 0.2392 |
| | 24 days | 38 | 21.6 | 155.0 | 72.28 | 62.25 | 31.989 | 5.189 | |
| | ALL | 74 | 18.8 | 155.0 | 68.59 | 62.60 | 27.552 | 3.203 | |
| TBG | 21 days | 36 | 23.7 | 61.3 | 45.72 | 45.35 | 8.202 | 1.367 | 0.7823 |
| | 24 days | 38 | 34.5 | 60.3 | 45.81 | 45.10 | 6.252 | 1.014 | |
| | ALL | 74 | 23.7 | 61.3 | 45.76 | 45.20 | 7.216 | 0.839 | |
| CBG | 21 days | 36 | 23.7 | 61.3 | 45.72 | 45.35 | 8.202 | 1.367 | 0.9584 |
| | 24 days | 38 | 34.5 | 60.3 | 45.81 | 45.10 | 6.252 | 1.014 | |
| | ALL | 74 | 23.7 | 61.3 | 45.76 | 45.20 | 7.216 | 0.839 | |

TABLE 15
Progesterone concentration at screening (ITT)

| Variable | Regimen | N | MIN | MAX | MEAN | MEDIAN | SD | SEM | p Value |
|---|---|---|---|---|---|---|---|---|---|
| PROGESTERONE | 21 days | 37 | 3 | 38 | 12.9 | 11.5 | 7.70 | 1.27 | 0.9943 |
| (ng/ml) | 24 days | 40 | 4 | 32 | 12.9 | 12.6 | 6.03 | 0.95 | |
| | ALL | 77 | 3 | 38 | 12.9 | 12.2 | 6.84 | 0.78 | |

Per Protocol Population Demographic and Baseline Characteristics

Overall, the 65 subjects of the PP population were 19-39 years of age (mean: 27.5 years), 70.8% were Caucasian, 27.7% Black and 1.5% Asian. The two regimen groups significantly (p=0.015) differed with respect to their mean age, which was 3 years lower in the 21-day regimen group (Table 16). There was no significant difference across regimen groups concerning the ethnic origin (Table 16).

TABLE 16
Demographic Characteristics, PP Population

| | 21-day regimen | 24-day regimen | |
|---|---|---|---|
| Characteristics | (N = 32) | (N = 33) | P-value |
| Age (years) | | | |
| Mean ± SD | 26.0 ± 4.8 | 29.0 ± 4.9 | 0.015 |
| Range | 19-38 | 20-38 | |
| Race | | | |
| Caucasian n (%) | 20 (62.5) | 26 (78.8) | 0.130 |
| Black n (%) | 12 (37.5) | 6 (18.2) | |
| Asian n (%) | 0 | 1 (3.0) | |

There were no significant difference between regimen groups in the mean age at menarche, mean duration of last menstruation cycle, gravidity and parity, and use of tobacco (less than 10 cigarettes per day as required by the protocol) (Table 17). For all women, the mean age at menarche was 12.7 years (range 9-16 years), the mean duration of last menstrual cycle was 28.6 days (range: 25-32 days) 52.3% were nulligravid, 78.5% were nulliparous and 41.5% smoked.

TABLE 17
Gynecological History and Tobacco Use, PP Population

| | 21-day regimen | 24-day regimen | |
|---|---|---|---|
| | (N = 32) | (N = 33) | P-value |
| Age at menarche (years) | | | |
| Mean ± SD | 12.6 ± 1.5 | 12.8 ± 1.3 | 0.711 |
| Range | 9-16 | 10-16 | |
| Duration of last menstrual | | | |
| cycle (days) | | | |
| Mean ± SD | 28.8 ± 1.6 | 28.4 ± 1.3 | 0.327 |
| Range | 25-32 | 25-32 | |
| Nulligravid n (%) | 18 (56.3) | 16 (48.5) | 0.531 |
| Nulliparous n (%) | 26 (81.3) | 25 (75.8) | 0.590 |
| Tobacco n (%) | 16 (50.0) | 11 (33.3) | 0.213 |

There were no remarkable differences across regimen groups in the proportions of subjects with medical histories and/or concomitant diseases and of subjects taking allowed concomitant therapy (Table 18 and Table 19).

TABLE 18
Medical history and/or concomitant diseases (PP)

| MEDICAL | | | | | | | |
|---|---|---|---|---|---|---|---|
| HISTORY/ | | | | | | | |
| CONCOMITANT | TOTAL | | 21 days | | 24 days | | p |
| DISEASES | N | % | N | % | N | % | Value |
| NO | 9 | 13.85 | 2 | 6.25 | 7 | 21.21 | 0.1487 |
| YES | 56 | 86.15 | 30 | 93.75 | 26 | 78.79 | |
| TOTAL | 65 | 100.00 | 32 | 100.00 | 33 | 100.00 | |

TABLE 19
Concomitant therapy (PP)

| CONCOMITANT | TOTAL | | 21 days | | 24 days | | |
|---|---|---|---|---|---|---|---|
| THERAPY | N | % | N | % | N | % | p Value |
| NO | 44 | 67.69 | 23 | 71.88 | 21 | 63.64 | 0.5977 |
| YES | 21 | 32.31 | 9 | 28.13 | 12 | 36.36 | |
| TOTAL | 65 | 100.00 | 32 | 100.00 | 33 | 100.00 | |

Subjects in the two regimen groups were not significantly different with respect to their mean weight, body mass index, or systolic and diastolic blood pressure (Table 20).

TABLE 20
Physical Examination, PP Population

| | 21-day regimen | 24-day regimen | |
|---|---|---|---|
| | (N = 32) | (N = 33) | P-value |
| Weight (kg)) | | | |
| Mean ± SD | 60.6 ± 8.6 | 61.4 ± 8.5 | 0.701 |
| Range | 48-82 | 50-78 | |
| BMI (kg/m²) | | | |
| Mean ± SD | 22.4 ± 2.9 | 22.7 ± 3.2 | 0.714 |
| Range | 17-29 | 18-30 | |
| Bystolic blood pressure | | | |
| (mmHg) | | | |
| Mean ± SD | 116.2 ± 9.9 | 116.1 ± 10.5 | 0.950 |
| Range | 94-137 | 101-145 | |
| Diastolic blood pressure | | | |
| (mmHg) | | | |
| Mean ± SD | 63.7 ± 6.5 | 63.0 ± 6.3 | 0.694 |
| Range | 46-77 | 52-79 | |

The gynecological examination, the characteristics of the cervical mucus evaluated with the Insler Score and the Pap smears were comparable across regimen groups (Table 21 to Table 23). There were only few abnormal findings at the gynecological examination and on Pap smears, which were not considered as clinically significant.

TABLE 21
Gynecological examination (PP)

| | | TOTAL | | 21 days | | 24 days | | p |
|---|---|---|---|---|---|---|---|---|
| | | N | % | N | % | N | % | Values |
| VULVA | NORMAL | 65 | 100.00 | 32 | 100.00 | 33 | 100.00 | — |
| | TOTAL | 65 | 100.00 | 32 | 100.00 | 33 | 100.00 | |
| VAGINAL | NORMAL | 64 | 98.46 | 31 | 96.88 | 33 | 100.00 | 0.4923 |
| | ABNORMAL NOT CS* | 1 | 1.54 | 1 | 3.13 | | | |
| | TOTAL | 65 | 100.00 | 32 | 100.00 | 33 | 100.00 | |
| CERVIX | NORMAL | 63 | 96.92 | 31 | 96.88 | 32 | 96.97 | 1.000 |
| | ABNORMAL NOT CS* | 2 | 3.08 | 1 | 3.13 | 1 | 3.03 | |
| | TOTAL | 65 | 100.00 | 32 | 100.00 | 33 | 100.00 | |
| UTERUS | NORMAL | 65 | 100.00 | 32 | 100.00 | 33 | 100.00 | — |
| | TOTAL | 65 | 100.00 | 32 | 100.00 | 33 | 100.00 | |
| OVARY | NORMAL | 65 | 100.00 | 32 | 100.00 | 33 | 100.00 | — |
| | TOTAL | 65 | 100.00 | 32 | 100.00 | 33 | 100.00 | |
| BREAST | NORMAL | 64 | 98.46 | 31 | 96.88 | 33 | 100.00 | 0.4923 |
| | ABNORMAL NOT CS* | 1 | 1.54 | 1 | 3.13 | | | |
| | TOTAL | 65 | 100.00 | 32 | 100.00 | 33 | 100.00 | |

*CS: Clinically significant

TABLE 22
Insler score (PP)

| | TOTAL | | 21 days | | 24 days | | |
|---|---|---|---|---|---|---|---|
| INSLER SCORE | N | % | N | % | N | % | p Value |
| 1 | 1 | 1.54 | | | 1 | 3.03 | 0.4121 |
| 2 | 4 | 6.15 | 1 | 3.13 | 3 | 9.09 | |
| 3 | 4 | 6.15 | 2 | 6.25 | 2 | 6.06 | |
| 4 | 3 | 4.62 | 2 | 6.25 | 1 | 3.03 | |
| 5 | 9 | 13.85 | 6 | 18.75 | 3 | 9.09 | |
| 6 | 11 | 16.92 | 8 | 25.00 | 3 | 9.09 | |
| 7 | 5 | 7.69 | 3 | 9.38 | 2 | 6.06 | |
| 8 | 6 | 9.23 | 1 | 3.13 | 5 | 15.15 | |
| 9 | 22 | 33.85 | 9 | 28.13 | 13 | 39.39 | |
| TOTAL | 65 | 100.00 | 32 | 100.00 | 33 | 100.00 | |

TABLE 23
Pap smear (PP)

| | TOTAL | | 21 days | | 24 days | | |
|---|---|---|---|---|---|---|---|
| PAP SMEAR | N | % | N | % | N | % | p Value |
| NORMAL | 63 | 96.92 | 31 | 96.88 | 32 | 96.97 | 1.000 |
| ABNORMAL NOT | 1 | 1.54 | | | 1 | 3.03 | |
| CS* | | | | | | | |
| INADEQUACY | 1 | 1.54 | 1 | 3.13 | | | |
| TOTAL | 65 | 100.00 | 32 | 100.00 | 33 | 100.00 | |

*CS: Clinically significant

Subjects in the two regimen groups were not significantly different with respect to findings at baseline endovaginal ultrasound. Overall there were 30.8 and 16.9% of women with at least one follicle more than 10 and 13 mm in diameter respectively. The mean diameter of the largest follicle was 8.4±4.37 mm (Table 24 and Table 25).

TABLE 24
Endovaginal ultrasound (PP)

| | | | | | | | | | p |
|---|---|---|---|---|---|---|---|---|---|
| Varibles | Regimen | N | MIN | MAX | MEAN | MEDIAN | SD | SEM | Values |
| UTERUS LENGTH | 21 days | 32 | 51 | 74 | 60.8 | 61.0 | 5.27 | 0.93 | 0.5924 |
| | 24 days | 33 | 46 | 78 | 59.9 | 59.0 | 7.94 | 1.38 | |
| | ALL | 65 | 46 | 78 | 60.3 | 60.0 | 6.72 | 0.83 | |
| UTERUS WIDTH | 21 days | 32 | 29 | 59 | 41.0 | 41.5 | 7.28 | 1.29 | 0.5722 |
| | 24 days | 33 | 27 | 66 | 42.1 | 40.0 | 8.97 | 1.56 | |
| | ALL | 65 | 27 | 66 | 41.6 | 41.0 | 8.14 | 1.01 | |
| UTERUS THICKNESS | 21 days | 32 | 24 | 44 | 33.9 | 34.0 | 5.11 | 0.90 | 0.4459 |
| | 24 days | 33 | 24 | 49 | 32.9 | 34.0 | 5.36 | 0.93 | |
| | ALL | 65 | 24 | 49 | 33.4 | 34.0 | 5.22 | 0.65 | |
| ENDOMETRIAL THICKNESS | 21 days | 32 | 4 | 14 | 8.2 | 8.0 | 2.19 | 0.39 | 0.0945 |
| | 24 days | 33 | 2 | 13 | 7.2 | 7.0 | 2.56 | 0.45 | |
| | ALL | 65 | 2 | 14 | 7.6 | 7.0 | 2.42 | 0.30 | |
| RIGHT OVARY DIAMETER | 21 days | 32 | 20 | 46 | 31.2 | 30.0 | 6.27 | 1.11 | 0.2398 |
| | 24 days | 33 | 17 | 41 | 29.5 | 29.0 | 5.71 | 0.99 | |
| | ALL | 65 | 17 | 46 | 30.3 | 29.0 | 6.01 | 0.75 | |
| LEFT OVARY DIAMETER | 21 days | 32 | 23 | 43 | 30.7 | 29.0 | 5.44 | 0.96 | 0.6471 |
| | 24 days | 33 | 20 | 41 | 30.1 | 29.0 | 5.56 | 0.97 | |
| | ALL | 65 | 20 | 43 | 30.4 | 29.0 | 5.47 | 0.68 | |

TABLE 25
Endovaginal ultrasound - Follicles (PP)

| | TOTAL | | 21 days | | 24 days | | |
|---|---|---|---|---|---|---|---|
| PRESENCE OF FOLLICLE | N | % | N | % | N | % | p Value |
| NO | 4 | 6.15 | 2 | 6.25 | 2 | 6.06 | 1.000 |
| YES | 61 | 93.85 | 30 | 93.75 | 31 | 93.94 | |
| TOTAL | 65 | 100.00 | 32 | 100.00 | 33 | 100.00 | |

| | | | | | | | | | p |
|---|---|---|---|---|---|---|---|---|---|
| Variable | Regimen | N | MIN | MAX | MEAN | MEDIAN | SD | SEM | Value |
| DIAMETER OF THE | 21 days | 32 | 0 | 19 | 9.1 | 8.5 | 4.74 | 0.84 | 0.2205 |
| LARGEST FOLLICLE | 24 days | 33 | 0 | 16 | 7.8 | 7.0 | 3.95 | 0.69 | |
| | ALL | 65 | 0 | 19 | 8.4 | 8.0 | 4.37 | 0.54 | |

| WOMEN WITH DIAMETER OF THE | TOTAL | | 21 days | | 24 days | | p |
|---|---|---|---|---|---|---|---|
| LARGEST FOLLICLE > 10 mm | N | % | N | % | N | % | Value |
| NO | 45 | 69.23 | 20 | 62.50 | 25 | 75.76 | 0.2469 |
| YES | 20 | 30.77 | 12 | 37.50 | 8 | 24.24 | |
| TOTAL | 65 | 100.00 | 32 | 100.00 | 33 | 100.00 | |

| | | | |
|---|---|---|---|
| NUMBER OF FOLLICLES | TOTAL | 21 days | 24 days |

| WITH DIAMETER > 10 mm | N | % | N | % | N | % |
|---|---|---|---|---|---|---|
| 0 | 45 | 69.23 | 20 | 62.50 | 25 | 75.76 |
| 1 | 19 | 29.23 | 11 | 34.38 | 8 | 24.24 |
| 2 | 1 | 1.54 | 1 | 3.13 | | |
| TOTAL | 65 | 100.00 | 32 | 100.00 | 33 | 100.00 |

| WOMEN WITH DIAMETER OF THE | TOTAL | | 21 days | | 24 days | | p |
|---|---|---|---|---|---|---|---|
| LARGEST FOLLICLE > 13 mm | N | % | N | % | N | % | Value |
| NO | 54 | 83.08 | 25 | 78.13 | 29 | 87.88 | 0.2944 |
| YES | 11 | 16.92 | 7 | 21.88 | 4 | 12.12 | |
| TOTAL | 65 | 100.00 | 32 | 100.00 | 33 | 100.00 | |

At baseline, pituitary and ovary hormones (LH, FSH, estradiol and progesterone) and carrier proteins (Sex Hormone Binding Globulin (SHBG), Cortisol Binding Globulin (CBG) and Thyroid Binding Globulin (TBG)), measured at Day 20 of the pre-treatment cycle were similar across regimen groups (Table 26 to Table 27). As requested by the protocol, all women had ovulation in the pre-treatment cycle, as assessed by a progesterone blood level >3 ng/ml (Table 27).

TABLE 26
Pituitary and ovary hormones and carrier proteins (PP)

| | | | | | | | | | p |
|---|---|---|---|---|---|---|---|---|---|
| Variable | Regimen | N | MIN | MAX | MEAN | MEDIAN | SD | SEM | Values |
| FSH | 21 days | 31 | 1.7 | 15.1 | 3.81 | 3.10 | 2.603 | 0.468 | 0.2612 |
| | 24 days | 32 | 1.5 | 14.5 | 4.58 | 3.76 | 2.726 | 0.482 | |
| | ALL | 63 | 1.5 | 15.1 | 4.20 | 3.51 | 2.673 | 0.337 | |
| LH | 21 days | 31 | 0.6 | 78.8 | 7.27 | 3.02 | 14.036 | 2.521 | 0.7091 |
| | 24 days | 32 | 0.2 | 32.7 | 6.22 | 3.91 | 7.277 | 1.286 | |
| | ALL | 63 | 0.2 | 78.8 | 6.74 | 3.70 | 11.049 | 1.392 | |
| PROGESTERONE | 21 days | 31 | 0.1 | 22.0 | 9.52 | 8.63 | 7.016 | 1.260 | 0.8680 |
| | 24 days | 32 | 0.5 | 23.8 | 9.79 | 9.58 | 6.061 | 1.071 | |
| | ALL | 63 | 0.1 | 23.8 | 9.66 | 9.04 | 6.497 | 0.818 | |
| E1 (estrone) | 21 days | 31 | 58.3 | 448.0 | 174.80 | 140.00 | 88.038 | 15.812 | 0.3436 |
| | 24 days | 32 | 76.1 | 325.0 | 154.88 | 125.50 | 77.392 | 13.681 | |
| | ALL | 63 | 58.3 | 448.0 | 164.68 | 139.00 | 82.740 | 10.424 | |
| E2 (estradiol) | 21 days | 31 | 92.0 | 447.0 | 221.62 | 197.00 | 90.985 | 16.341 | 0.6377 |
| | 24 days | 32 | 51.4 | 522.0 | 210.42 | 186.00 | 96.661 | 17.087 | |
| | ALL | 63 | 51.4 | 522.0 | 215.93 | 196.00 | 93.323 | 11.758 | |
| SHBG | 21 days | 31 | 18.8 | 99.8 | 62.10 | 63.20 | 18.878 | 3.391 | 0.0551 |
| | 24 days | 32 | 21.6 | 155.0 | 75.64 | 67.50 | 33.782 | 5.972 | |
| | ALL | 63 | 18.8 | 155.0 | 68.98 | 64.30 | 28.101 | 3.540 | |
| CBG | 21 days | 31 | 23.7 | 61.3 | 45.16 | 45.20 | 8.089 | 1.453 | 0.5458 |
| | 24 days | 32 | 34.5 | 60.3 | 46.28 | 45.25 | 6.571 | 1.162 | |
| | ALL | 63 | 23.7 | 61.3 | 45.73 | 45.20 | 7.320 | 0.922 | |
| TBG | 21 days | 31 | 16.1 | 28.6 | 22.17 | 22.30 | 3.009 | 0.540 | 0.6211 |
| | 24 days | 32 | 15.7 | 29.7 | 21.81 | 22.05 | 2.870 | 0.507 | |
| | ALL | 63 | 15.7 | 29.7 | 21.99 | 22.10 | 2.921 | 0.368 | |

TABLE 27
Progesterone concentration at screening

| Variable | Regimen | N | MIN | MAX | MEAN | MEDIAN | SD | SEM | p Value |
|---|---|---|---|---|---|---|---|---|---|
| PROGESTERONE | 21 days | 32 | 3 | 38 | 12.4 | 11.2 | 7.86 | 1.39 | 0.9099 |
| | 24 days | 33 | 4 | 32 | 12.2 | 11.3 | 6.11 | 1.06 | |
| | ALL | 65 | 3 | 38 | 12.3 | 11.3 | 6.97 | 0.86 | |

Measurement of Treatment Compliance

The compliance with the dosing regimen was checked from the information provided in subject's diaries. To verify compliance with the treatment, NOMAC blood levels were measured in all blood samples after the end of the study. E2 levels were measured at the same time. Measurements were performed using a liquid chromatography-mass spectrometry/mass spectrometry (LC-MS/MS) validated method.

From this data, treatment compliance was defined as follows: a compliant cycle was any cycle fulfilling the conditions that (i) no pills are missed from Day 1 to Day 24 (inclusive) or no more than one dose was missed in this period, provided the subject took two doses the day after, and (ii) no NOMAC serum level was below the limit of quantification during the active treatment; a compliant subject was any subject compliant during all treatment cycles.

Table 28 presents the mean NOMAC and E2 blood levels during treatment cycles, obtained from all measurements performed while the subjects took the active treatment.

TABLE 28
Mean NOMAC and E2 blood levels during treatment cycles in the ITT
population

| | | 21-day regimen | 24-day regimen | |
|---|---|---|---|---|
| | Cycle | m ± SD | m ± SD | P-value |
| E2 | Cycle 1* | 74.9 ± 46.92 | 87.7 ± 57.39 | 0.300 |
| (pg/ml) | Cycle 2 | 97.8 ± 40.42 | 88.6 ± 39.92 | 0.331 |
| | Cycle 3 | 122.4 ± 98.29 | 88.7 ± 43.74 | 0.069 |
| | All | 106.4 ± 58.68 | 88.7 ± 39.84 | 0.131 |
| Nomac | Cycle 1* | 4.1 ± 1.66 | 4.7 ± 1.84 | 0.187 |
| (ng/ml) | Cycle 2 | 3.9 ± 1.28 | 3.9 ± 1.09 | 0.766 |
| | Cycle 3 | 4.0 ± 1.46 | 4.0 ± 1.27 | 0.799 |
| | All | 3.9 ± 1.32 | 4.0 ± 1.16- | 0.797 |

*measured only on Day 21

For each parameter there were no significant difference among regimens and cycles.

Efficacy Results

Ovarian Activity from Ultrasound Assessments

Table 29 gives the percentage of women with at least one follicle >10 mm and >13 mm in diameter during the treatment period in the PP and in the ITT population.

TABLE 29
Incidence of follicle >10 mm and >13 mm in diameter

| | | 21-day regimen | 24-day regimen | |
|---|---|---|---|---|
| Population | Diameter | n (%) | n (%) | P-value |
| ITT | >10 mm | 19 (51.4) | 13 (33.3) | 0.112 |
| | >13 mm | 12 (32.4) | 6 (15.4) | 0.081 |
| PP | >10 mm | 15 (46.9) | 9 (27.3) | 0.102 |
| | >13 mm | 8 (25.0) | 5 (15.2) | 0.321 |

There were no statistical differences between the two regimen groups. Nevertheless there were in the 24-day regimen group about half fewer women with a follicle >13 mm than in the 21-day regimen. In each group there were 3 women with more than one follicle >10 mm (Table 30).

TABLE 30
Incidence of follicle > 10 mm and > 13 mm in diameter (ITT)

| | | | 21 Days | | 24 Days | | |
|---|---|---|---|---|---|---|---|
| | TOTAL | | Regimen | | Regimen | | |
| | N | % | N | % | N | % | p Value |
| AT LEAST ONE | | | | | | | |
| FOLLICLE > | | | | | | | |
| 10 mm | | | | | | | |
| NO | 44 | 57.89 | 18 | 48.65 | 26 | 66.67 | 0.1118 |
| YES | 32 | 42.11 | 19 | 51.35 | 13 | 33.33 | |
| TOTAL | 76 | 100.00 | 37 | 100.00 | 39 | 100.00 | |
| AT LEAST ONE | | | | | | | |
| FOLLICLE > | | | | | | | |
| 13 mm | | | | | | | |
| NO | 58 | 76.32 | 25 | 67.57 | 33 | 84.62 | 0.0806 |
| YES | 18 | 23.68 | 12 | 32.43 | 6 | 15.38 | |
| TOTAL | 76 | 100.00 | 37 | 100.00 | 39 | 100.00 | |
| AT LEAST ONE | | | | | | | |
| ULTRA- | | | | | | | |
| SONOGRAPHY | | | | | | | |
| WITH MORE | | | | | | | |
| THAN ONE | | | | | | | |
| FOLLICLE > | | | | | | | |
| 10 mm | | | | | | | |
| NO | 70 | 92.11 | 34 | 91.89 | 36 | 92.31 | 1.000 |
| YES | 6 | 7.89 | 3 | 8.11 | 3 | 7.69 | |
| TOTAL | 76 | 100.00 | 37 | 100.00 | 39 | 100.00 | |

Table 31 and Table 32 give the mean diameter of the largest follicle during the treatment period in the PP and in the ITT population respectively.

TABLE 31
Mean diameter (mm) of the largest follicle in the ITT population

| Treatment | 21-day regimen | 24-day regimen | |
|---|---|---|---|
| cycle | m ± SD | m ± SD | P-value |
| Cycle 1 | 8.6 ± 5.75 | 6.9 ± 2.28 | 0.078 |
| Cycle 2 | 11.3 ± 5.33 | 9.0 ± 3.00 | 0.020 |
| Cycle 3 | 11.5 ± 6.04 | 9.2 ± 3.04 | 0.041 |
| All | 13.0 ± 7.52 | 9.9 ± 3.36 | 0.024 |

TABLE 32
Mean diameter (mm) of the largest follicle in the PP population

| Treatment | 21-day regimen | 24-day regimen | |
|---|---|---|---|
| cycle | m ± SD | m ± SD | P-value |
| Cycle 1 | 8.3 ± 4.66 | 6.8 ± 2.24 | 0.074 |
| Cycle 2 | 10.7 ± 4.04 | 9.0 ± 3.06 | 0.045 |
| Cycle 3 | 10.5 ± 3.73 | 9.1 ± 3.01 | 0.041 |
| All | 11.4 ± 4.16 | 9.7 ± 3.45 | 0.081 |

In the two populations the mean diameter of the largest follicle in the 24-day regimen group was lower than in the 21-day regimen group. The difference between the 2 groups was statistically significant for cycle 2 and cycle 3 in the two populations, and for all treatment cycles considered as a whole in the ITT population.

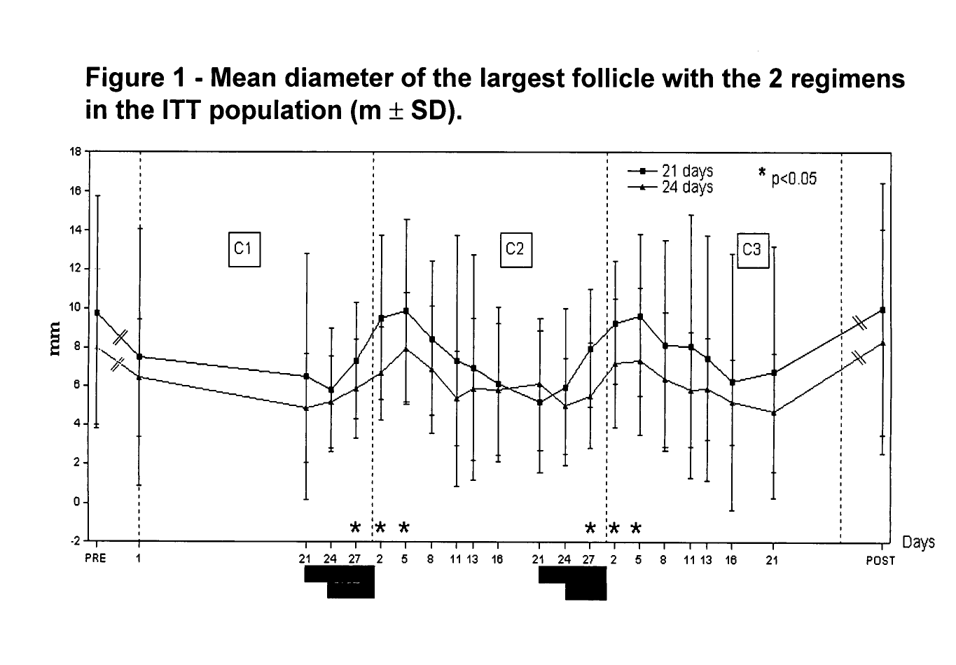

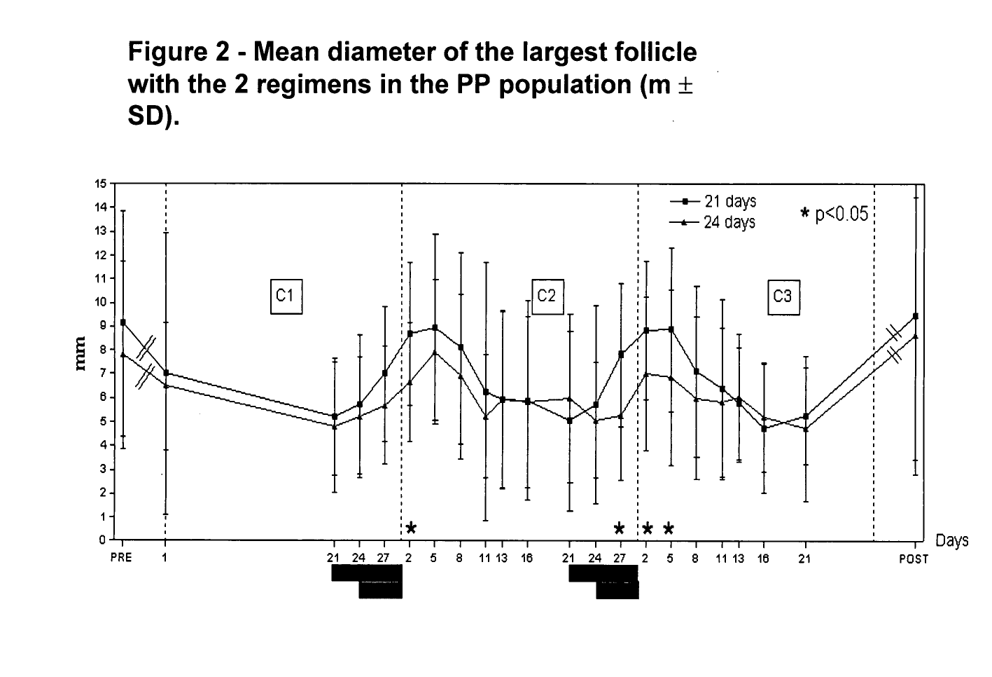

The mean diameters of the largest follicle at each assessment during the study for the two regimens are shown in FIG. 1 and in FIG. 2 for the ITT and PP population respectively.

The change in the diameter of the largest follicle was similar in the two populations. The mean diameter for the 24-day regimen remained at <8 mm throughout the 3 treatment cycles; with the 21-day regimen, the mean diameter rose near to 10 mm in treatment cycles 2 and 3. The mean diameter of the largest follicle was generally found significantly lower in the 24-day regimen groups at the assessment performed at the end of each pill free interval (day 27) and at the beginning of each following treatment cycle (day 2 and 5).

Hormone Assessments

Progesterone Levels

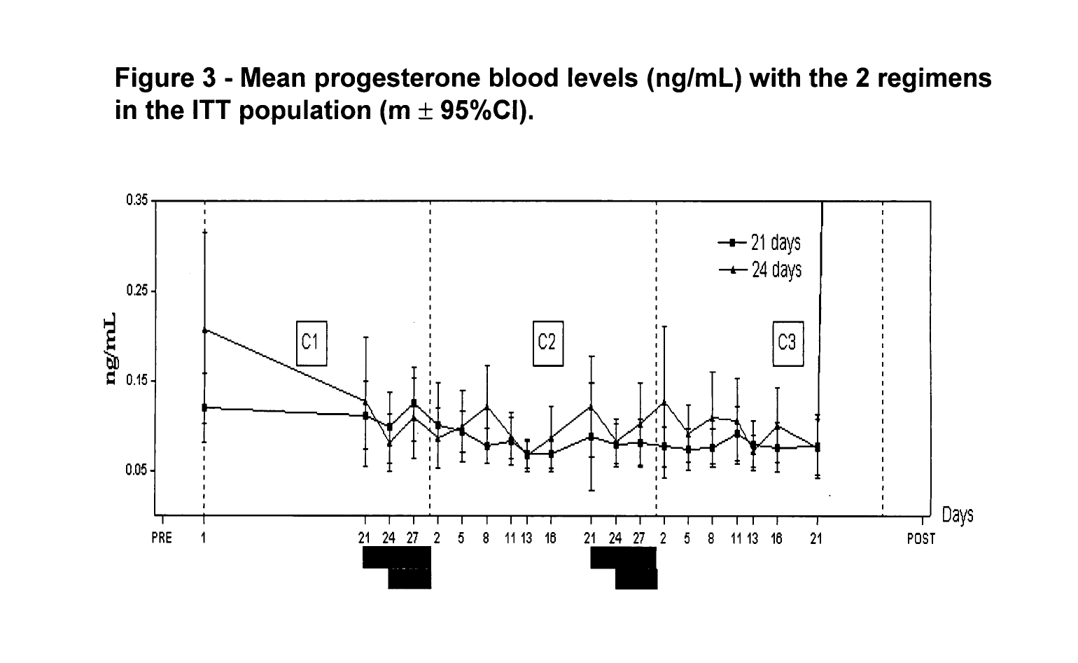

During the treatment period, there were no progesterone blood levels ≧3 ng/ml in the two populations with the two regimens. That means that there was no ovulation or luteal unruptured follicle syndrome during the study. As shown in FIG. 3, the mean progesterone levels remained below 0.15 ng/ml throughout the 3 treatment cycles in the two groups.

Estradiol Levels

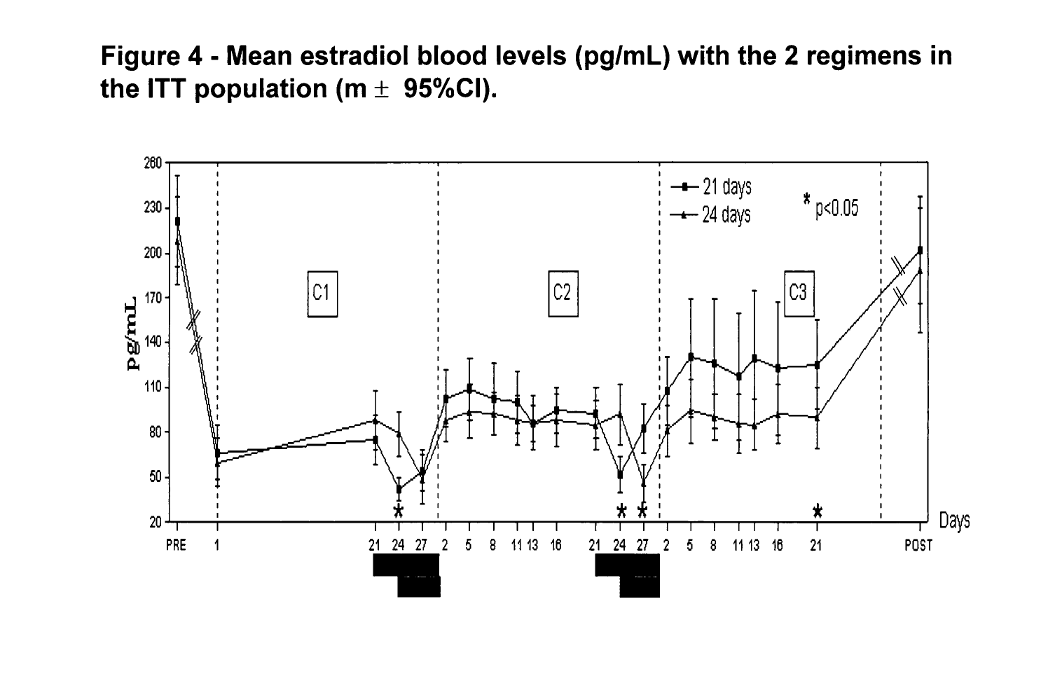

FIG. 4 shows the mean estradiol blood levels at each assessment throughout the study. There was a statistically significant difference between the 2 regimen groups at four assessments. At day 24 of treatment cycles 1 and 2, the mean estradiol level was significantly higher in the 24-day regimen groups (last day of active treatment) than in the 21-day regimen group (third day of the pill-free interval). At the end of the second pill-free interval (day 27), the mean estradiol level was significantly lower in the 24-day regimen groups, compared to the 21-day regimen group. The difference between the 2 groups remained throughout the following treatment cycle (cycle 3) but was statistically significant only at day 21. Changes in estrone levels were quite similar.

FSH Levels

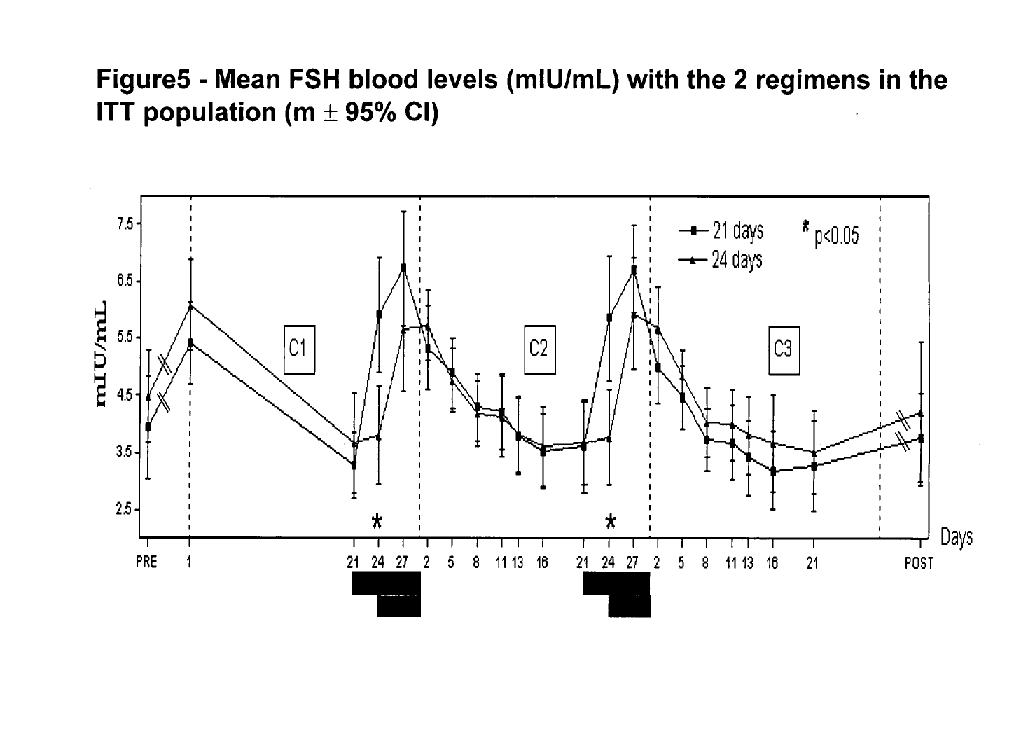

The mean blood levels of FSH at each assessment are given in FIG. 5. In the 21-day regimen group, there was, after the end of the active treatment, a rapid and dramatic increase in FSH. This increase was delayed and a little bit lower in the 24-day regimen groups. Nevertheless the mean FSH level was found significantly lower only at day 24 with the 24-day regimen.

LH Levels

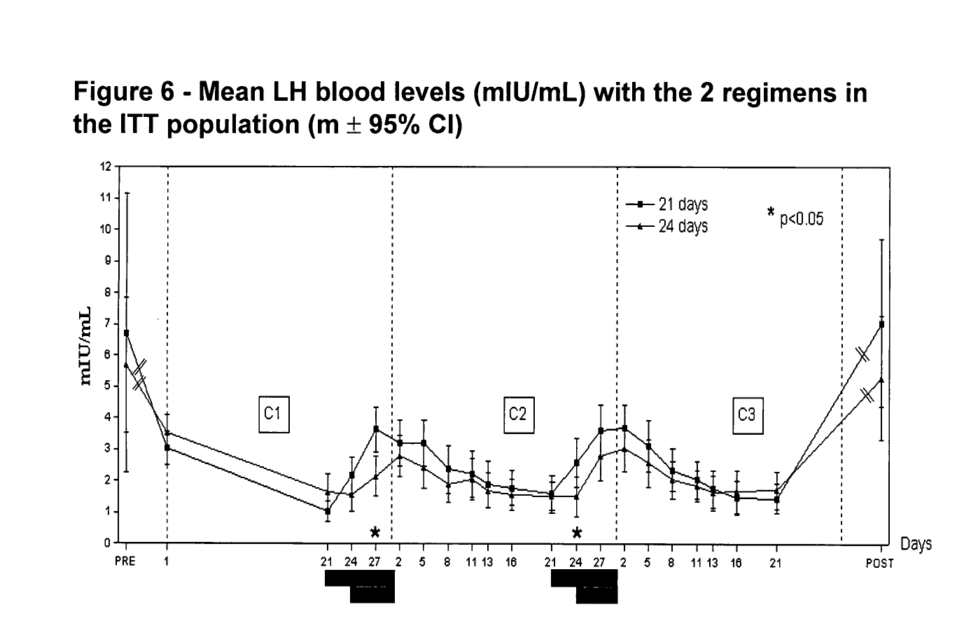

The mean blood levels of LH at each assessment are given in FIG. 6. It can be checked that there were no LH ovulatory peaks during the treatment period with the 2 regimens. The mean LH levels remained below 4 mIU/ml throughout the treatment cycles. They were lower in the 24-day regimen groups but the difference with the 21-day regimen group was statistically significant once during each pill free interval: at day 27 of treatment cycle 1 and 2, at day 24 of treatment cycle 2. The results obtained for the PP population were quite similar.

Bleeding Pattern

The analyses of genital bleeding were performed from the data recorded in the menstrual diaries. Two subjects who failed to return a diary were excluded from bleeding pattern analyses. The data presented hereunder are given for the ITT population. The results obtained for the PP population were similar.

Duration of Genital Bleeding

Table 33 summarizes the duration of genital bleeding during the treatment period, including the spontaneous menstruation occurring at the end of the pre-treatment cycle, the withdrawal bleedings occurring after treatment cycles 1 and 2 and all intermenstrual bleeding recorded between these three bleeding episodes.

TABLE 33
Number of days of bleeding during the treatment period in the ITT
population

| | 21-day regimen | 24-day regimen | |
|---|---|---|---|
| | m ± SD | m ± SD | P-value |
| Total duration | 15.5 ± 5.57 | 12.4 ± 4.87 | 0.013 |
| Last spontaneous | 4.1 ± 1.80 | 4.6 ± 3.18 | 0.383 |
| menstruation | | | |
| Withdrawal bleeding | | | |
| Cycle 1 | 5.0 ± 2.55 | 3.5 ± 1.29 | 0.002 |
| Cycle 2 | 4.8 ± 1.74 | 3.9 ± 1.55 | 0.030 |
| Intermenstrual bleeding | 2.4 ± 4.46 | 1.3 ± 2.98 | 0.207 |

The mean total duration of genital bleeding was statistically shorter of about 3 days with the 24-day regimen compared to 21-day regimen (12.4±4.87 versus 15.5±5.57 days, p<0.05). The difference between the two groups was due to a shorter duration of both intermenstrual bleeding and withdrawal bleeding with the 24-day regimen. Nevertheless only the difference for withdrawal bleedings reached statistical significance.

Withdrawal Bleeding

Table 34 summarizes the characteristics of withdrawal bleeding.

TABLE 34
Characteristics of withdrawal bleeding (wb) in the ITT population

| | 21-day regimen | 24-day regimen | P-value |
|---|---|---|---|
| Number of women | 36 | 39 | |
| Number of cycles | 107 | 115 | |
| Number of women with | 32 (88.9%) | 34 (87.2%) | 1.00 |
| wb at each cycle | | | |
| Number of cycles with wb | 102 (95.3%) | 108 (93.9%) | 0.642 |
| Time to onset, all cycles | 3.6 ± 3.30 | 4.5 ± 4.97 | 0.139 |
| (days) | | | |
| Duration, all cycles (days) | 4.9 ± 2.18 | 3.7 ± 1.43 | <0.001 |
| Intermenstrual duration | 26.7 ± 4.16 | 28.5 ± 5.59 | 0.011 |
| (days) | | | |

The percentage of women with withdrawal bleeding at the end of all treatment cycles was about 88%, and was not significantly different for the two regimens.

Across all cycles, the number of cycles with withdrawal bleeding (94 to 95%), the mean time from day of last active treatment to the onset of withdrawal bleeding (3.6 to 4.5 days), were not significantly different for the two regimen groups.

Among subjects with withdrawal bleeding at the end of cycles 1 and 2, the mean duration of withdrawal bleeding was statistically significant across regimen groups: 3.7±1.43 days with the 24-day regimen versus 4.9±2.18 days after the 21-day regimen (p=0.001) (Table 35). The mean intermenstrual duration (i.e. interval between the first day of two consecutive withdrawal bleedings) was near 28 days but significantly shorter in the 21-day regimen compared to the 24-day regimen (26.7 versus 28.5 days).

TABLE 35
Duration of withdrawal bleeding (ITT)

| | | | | | | | | | p |
|---|---|---|---|---|---|---|---|---|---|
| | Regimen | N | MIN | MAX | MEAN | MEDIAN | SD | SEM | Values |
| DURATION OF | | | | | | | | | |
| WITHDRAWAL BLEEDING | | | | | | | | | |
| pre-treatment cycle | 21 Days | 36 | 0 | 10 | 4.1 | 4.0 | 1.80 | 0.30 | 0.3832 |
| Last spontaneous | 24 Days | 39 | 1 | 18 | 4.6 | 4.0 | 3.18 | 0.51 | |
| menstruation | ALL | 75 | 0 | 18 | 4.4 | 4.0 | 2.61 | 0.30 | |
| Cycle 1 (C1) | 21 Days | 34 | 3 | 16 | 5.0 | 5.0 | 2.55 | 0.44 | 0.0022 |
| | 24 Days | 35 | 1 | 7 | 3.5 | 3.0 | 1.29 | 0.22 | |
| | ALL | 69 | 1 | 16 | 4.2 | 4.0 | 2.14 | 0.26 | |
| Cycle 2 (C2) | 21 Days | 32 | 2 | 11 | 4.8 | 5.0 | 1.74 | 0.31 | 0.0300 |
| | 24 Days | 34 | 1 | 7 | 3.9 | 4.0 | 1.55 | 0.27 | |
| | ALL | 66 | 1 | 11 | 4.3 | 4.0 | 1.69 | 0.21 | |
| MEAN DURATION FOR | 21 Days | 35 | 3.0 | 11.5 | 4.93 | 4.5 | 1.787 | 0.302 | 0.0010 |
| Cycle 1 and Cycle 2 | 24 Days | 36 | 1.0 | 6.0 | 3.68 | 3.5 | 1.196 | 0.199 | |
| | ALL | 71 | 1.0 | 11.5 | 4.30 | 4.0 | 1.631 | 0.194 | |
| MEAN DURATION OF | | | | | | | | | |
| WITHDRAWAL BLEEDING | | | | | | | | | |
| Cycle 1 and Cycle 2 | 21 Days | 66 | 2.0 | 16.0 | 4.91 | 5.0 | 2.182 | 0.269 | 0.0002 |
| | 24 Days | 69 | 1.0 | 7.0 | 3.68 | 4.0 | 1.430 | 0.172 | |
| | ALL | 135 | 1.0 | 16.0 | 4.28 | 4.0 | 1.930 | 0.166 | |

The first day of withdrawal bleeding occurred, in most cases, between day 23 and day 28 of the current cycle in the 21-day regimen group and between day 26 of the current cycle and day 2 of the next cycle in the 24-day regimen group (Table 36).

TABLE 36
Day of cycle corresponding to onset of withdrawal bleeding (ITT)

| | | | | 21 Days | | 24 Days | | |
|---|---|---|---|---|---|---|---|---|
| | | TOTAL | | Regimen | | Regimen | | p |
| Cycle | Day | N | % | N | % | N | % | Values |
| C1 | MV | 8 | 10.39 | 3 | 8.11 | 5 | 12.50 | <0.0001 |
| | C1_day 11 | 1 | 1.30 | 1 | 2.70 | | | |
| | C1_day 15 | 1 | 1.30 | 1 | 2.70 | | | |
| | C1_day 16 | 1 | 1.30 | 1 | 2.70 | | | |
| | C1_day 21 | 2 | 2.60 | 2 | 5.41 | | | |
| | C1_day 22 | 1 | 1.30 | | | 1 | 2.50 | |
| | C1_day 23 | 1 | 1.30 | 1 | 2.70 | | | |
| | C1_day 24 | 7 | 9.09 | 7 | 18.92 | | | |
| | C1_day 25 | 9 | 11.69 | 9 | 24.32 | | | |
| | C1_day 26 | 11 | 14.29 | 6 | 16.22 | 5 | 12.50 | |
| | C1_day 27 | 8 | 10.39 | 4 | 10.81 | 4 | 10.00 | |
| | C1_day 28 | 15 | 19.48 | 2 | 5.41 | 13 | 32.50 | |
| | C2_day 1 | 10 | 12.99 | | | 10 | 25.00 | |
| | C2_day 2 | 1 | 1.30 | | | 1 | 2.50 | |
| | C2_day 4 | 1 | 1.30 | | | 1 | 2.50 | |
| | TOTAL | 77 | 100.00 | 37 | 100.00 | 40 | 100.00 | |
| C2 | MV | 11 | 14.29 | 5 | 13.51 | 6 | 15.00 | <0.0001 |
| | C2_day 14 | 1 | 1.30 | 1 | 2.70 | | | |
| | C2_day 16 | 1 | 1.30 | 1 | 2.70 | | | |
| | C2_day 18 | 1 | 1.30 | | | 1 | 2.50 | |
| | C2_day 22 | 1 | 1.30 | 1 | 2.70 | | | |
| | C2_day 23 | 2 | 2.60 | 1 | 2.70 | 1 | 2.50 | |
| | C2_day 24 | 5 | 6.49 | 5 | 13.51 | | | |
| | C2_day 25 | 6 | 7.79 | 6 | 16.22 | | | |
| | C2_day 26 | 15 | 19.48 | 11 | 29.73 | 4 | 10.00 | |
| | C2_day 27 | 12 | 15.58 | 3 | 8.11 | 9 | 22.50 | |
| | C2_day 28 | 11 | 14.29 | 3 | 8.11 | 8 | 20.00 | |
| | C3_day 1 | 8 | 10.39 | | | 8 | 20.00 | |
| | C3_day 2 | 3 | 3.90 | | | 3 | 7.50 | |
| | TOTAL | 77 | 100.00 | 37 | 100.00 | 40 | 100.00 | |
| C3 | MV | 7 | 9.09 | 3 | 8.11 | 4 | 10.00 | <0.0001 |
| | C3_day 12 | 1 | 1.30 | | | 1 | 2.50 | |
| | C3_day 13 | 1 | 1.30 | 1 | 2.70 | | | |
| | C3_day 15 | 1 | 1.30 | | | 1 | 2.50 | |
| | C3_day 18 | 1 | 1.30 | 1 | 2.70 | | | |
| | C3_day 19 | 1 | 1.30 | 1 | 2.70 | | | |
| | C3_day 23 | 5 | 6.49 | 5 | 13.51 | | | |
| | C3_day 24 | 3 | 3.90 | 2 | 5.41 | 1 | 2.50 | |
| | C3_day 25 | 9 | 11.69 | 9 | 24.32 | | | |
| | C3_day 26 | 5 | 6.49 | 4 | 10.81 | 1 | 2.50 | |
| | C3_day 27 | 10 | 12.99 | 6 | 16.22 | 4 | 10.00 | |
| | C3_day 28 | 14 | 18.18 | 3 | 8.11 | 11 | 27.50 | |
| | C4_day 01 | 7 | 9.09 | 1 | 2.70 | 6 | 15.00 | |
| | C4_day 02 | 3 | 3.90 | | | 3 | 7.50 | |
| | C4_day 03 | 2 | 2.60 | 1 | 2.70 | 1 | 2.50 | |
| | C4_day 05 | 1 | 1.30 | | | 1 | 2.50 | |
| | C4_day 06 | 1 | 1.30 | | | 1 | 2.50 | |
| | C4_day 12 | 2 | 2.60 | | | 2 | 5.00 | |
| | C4_day 16 | 2 | 2.60 | | | 2 | 5.00 | |
| | C5_day 04 | 1 | 1.30 | | | 1 | 2.50 | |
| | TOTAL | 77 | 100.00 | 37 | 100.00 | 40 | 100.00 | |

Intermenstrual Bleeding

As shown in Table 37, the proportion of women with at least one day of intermenstrual bleeding and the percentage of treatment cycles with intermenstrual bleeding were not significantly different in the 2 regimen groups. The total duration of intermenstrual bleeding and the mean duration per cycle were shorter in the 24-day regimen groups but the difference between the two groups reached statistical significance only for the second parameter: there were with the 24-day regimen 2.4 fewer days of intermenstrual bleeding per cycle.

TABLE 37
Incidence and duration of intermenstrual bleeding (ib) in the ITT
population

| | 21-day regimen | 24-day regimen | P-value |
|---|---|---|---|
| Number of women | 36 | 39 | |
| Number of cycles | 107 | 115 | |
| Number of women with at | 13 (36.1%) | 13 (33.3%) | 0.804 |
| least one day of ib | | | |
| Number of cycles with at | 15 (14.2%) | 22 (19.3%) | 0.310 |
| least one day of ib | | | |
| Duration, all cycles | 6.6 ± 5.27 | 3.9 ± 4.18 | 0.095 |
| (days) | (n = 13) | (n = 13) | |
| Duration per cycle (days) | 5.7 ± 4.95 | 2.3 ± 2.19 | 0.021 |
| | (n = 15) | (n = 22) | |

Cervical Mucus

Table 38 presents the cervical mucus score measured during 4 cycles: pre-treatment cycle, treatment cycles 2 and 3, and post treatment cycle, for the 2 groups in the ITT population.

TABLE 38
Mean cervical mucus score at each assessment in the ITT population

| | 21-day regimen | 24-day regimen | |
|---|---|---|---|
| Cycle | (N = 37) | (N = 39) | P-value |
| Pre-treatment | 6.2 ± 2.20 | 6.9 ± 2.50 | 0.142 |
| Treatment cycle 2 | 1.6 ± 1.57 | 1.2 ± 1.16 | 0.378 |
| Treatment cycle 3 | 0.7 ± 1.13 | 0.9 ± 1.47 | 0.800 |
| Post treatment cycle | 5.2 ± 3.07 | 5.8 ± 2.55 | 0.508 |
| change from baseline to | <0.0001 | <0.0001 | — |
| cycle 2 | | | |
| p Value | | | |
| change from baseline to | <0.0001 | <0.0001 | — |
| cycle 3 | | | |
| p Value | | | |

The mean cervical mucus score was not significantly different between the 2 regimen groups at each assessment. Nevertheless there was a significantly difference across cycles. Compared to the pre-treatment value, the mean cervical mucus index decreased by 79 and 88% for all subjects during treatment cycles 2 and 3, respectively.

Endometrial Thickness

The mean endometrial thickness at each assessment (pre-treatment cycle, treatment cycle 3 and post treatment cycle) are given in Table 39.

TABLE 39
Mean endometrial thickness at each assessment in the ITT
population

| Cycle | 21-day regimen | 24-day regimen | P-value |
|---|---|---|---|
| Pre-treatment cycle | 7.9 ± 2.21 | 7.4 ± 2.45 | 0.288 |
| | (n = 37) | (n = 39) | |
| Treatment cycle 3 | 3.8 ± 3.8 | 3.6 ± 1.46 | 0.820 |
| | (n = 18) | (n = 18) | |
| Post treatment cycle | 6.5 ± 2.36 | 6.5 ± 1.86 | 0.979 |
| | (n = 35) | (n = 30) | |

At each assessment, there was no significant difference among the regimen groups. For all women, the endometrial thickness was reduced by half during treatment, compared to the pre-treatment value.

Return of Fertility

As previously shown in Table 38 and Table 39 the cervical mucus index and the endometrial thickness measured during the post treatment cycle returned back to the pre-treatment value.

A pregnancy occurred during the post treatment cycle in one woman (subject 001) who decided to abort.

Progesterone blood levels measured once in the second part of post treatment cycle was found ≧3 ng/ml (i.e corresponding to an ovulatory cycle) in 52 (72%) women (Table 40).

TABLE 40
Progesterone blood levels on post treatment cycle

| | | | 21 Days | | 24 Days | | |
|---|---|---|---|---|---|---|---|
| Progesterone > | TOTAL | | Regimen | | Regimen | | |
| 3 ng/ml | N | % | N | % | N | % | p Value |
| NO | 20 | 27.78 | 10 | 28.57 | 10 | 27.03 | 0.8837 |
| YES | 52 | 72.22 | 25 | 71.43 | 27 | 72.97 | |
| TOTAL | 72 | 100.00 | 35 | 100.00 | 37 | 100.00 | |

The occurrence of a menstrual bleeding was checked for all other women during the post treatment cycle (Table 41).

TABLE 41
Incidence of withdrawal bleeding (ITT)

| | | 21 Days | | 24 Days | | |
|---|---|---|---|---|---|---|
| TOTAL | | Regimen | | Regimen | | p |
| N | % | N | % | N | % | Values |

Number of WOMEN with
withdrawal bleeding

| | | | | | | | | |
|---|---|---|---|---|---|---|---|---|
| Cycle 1 | NO | 6 | 8.00 | 2 | 5.56 | 4 | 10.26 | 0.6759 |
| | YES | 69 | 92.00 | 34 | 94.44 | 35 | 89.74 | |
| | TOTAL | 75 | 100.00 | 36 | 100.00 | 39 | 100.00 | |
| Cycle 2 | NO | 6 | 8.33 | 3 | 8.57 | 3 | 8.11 | 1.000 |
| | YES | 66 | 91.67 | 32 | 91.43 | 34 | 91.89 | |
| | TOTAL | 72 | 100.00 | 35 | 100.00 | 37 | 100.00 | |
| Cycle 3 | YES | 71 | 100.00 | 34 | 100.00 | 37 | 100.00 | — |
| | TOTAL | 71 | 100.00 | 34 | 100.00 | 37 | 100.00 | |
| AT EACH CYCLE | NO | 9 | 12.00 | 4 | 11.11 | 5 | 12.82 | 1.000 |
| | YES | 66 | 88.00 | 32 | 88.89 | 34 | 87.18 | |
| | TOTAL | 75 | 100.00 | 36 | 100.00 | 39 | 100.00 | |

NUMBER OF CYCLES
WITH WITHDRAWAL
BLEEDING

| | | | | | | | |
|---|---|---|---|---|---|---|---|
| NO | 12 | 5.41 | 5 | 4.67 | 7 | 6.09 | 0.6415 |
| YES | 210 | 94.59 | 102 | 95.33 | 108 | 93.91 | |
| TOTAL | 222 | 100.00 | 107 | 100.00 | 115 | 100.00 | |

Tabulation of Individual Response Data

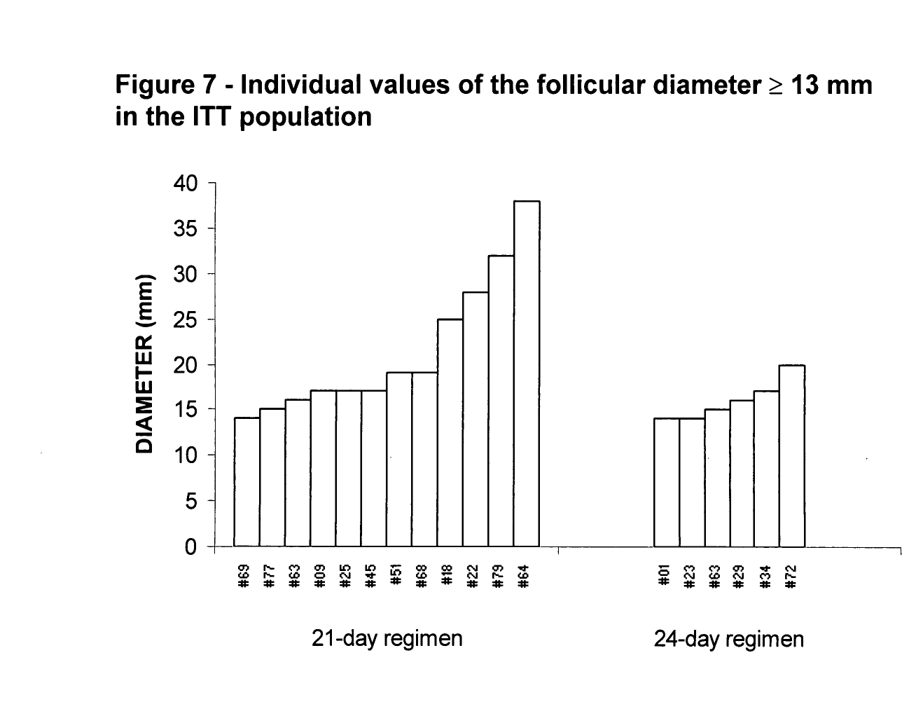

FIG. 7 shows the individual values of the follicular diameter for women with a follicle more than 13 mm diameter during treatment in each regimen group. Among the women who completed the study, there were 3 non-treatment compliant women in each group.

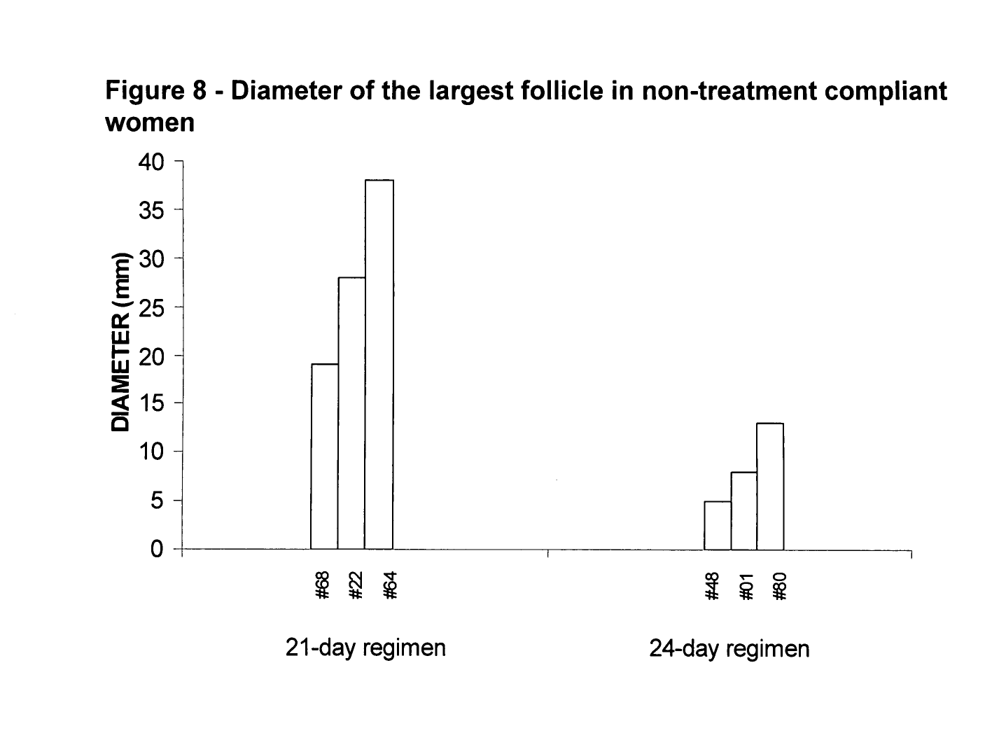

FIG. 8 presents for these women the diameter of the largest follicle measured during the corresponding non-compliant cycle. In the 24-day regimen group, the diameter of the largest follicle was not higher than 13 mm in non-compliant women. On the contrary it was higher than 13 mm in all non-compliant women of the 21-day regimen group.

In summary, in the two regimen groups there was no ovulation, nor LUF syndrome, and progesterone blood levels remained very low throughout the treatment period. Compared to the 21-day regimen, the 24-day regimen resulted in a significantly stronger inhibition of follicular growth. This effect was illustrated by the statistically lower diameter of the largest follicle at the end of the pill-free interval and at the beginning of the consecutive cycle. The lowest estradiol blood levels found at the end of the second pill-free interval and during treatment cycle 3 in the 24-day regimen group could also account for the stronger inhibition of follicular growth. The 24-day regimen delayed the increase in FSH during the pill-free interval. LH and FSH were found significantly lower with this regimen, at least at one measurement in each pill-free interval. The 24-day regimen also resulted in a better bleeding pattern. The total number of genital bleeding days was found significantly lower than with the 21-day regimen. The bleeding duration was shorter for both withdrawal and intermenstrual bleeding/spotting but the difference reached statistical significance only for withdrawal bleeding. There were no significant differences between the two groups concerning the incidence of intermenstrual bleeding, but the duration of intermenstrual bleeding per cycle was significantly shorter with the 24-day regimen. The two regimens were similarly able to decrease the cervical mucus index and the endometrial thickness. Lastly, return of fertility was proven in all women during the post treatment cycle.

Discussion

In the Regimen Validation Study, the same contraceptive combination (E2 1.5 mg/NOMAC 2.5 mg) was randomly given in two regimens: 21 and 24 out of 28 days for 3 consecutive treatment cycles. Medication was identical for the two treatment groups (i.e. appearance of active and placebo tables was identical for both treatment groups), i.e, women were not aware of being randomized to either 21-7 or 24-4 (double-blinded study design).

In the present study, there was no ovulation, nor LUF syndrome in the two tested regimens. The blood progesterone levels remained very low throughout the study period in both groups.

Nevertheless the monitoring of follicular maturation by vaginal ultrasound found some significant differences between the 2 groups. Giving the contraceptive combination for 24 versus 21 days resulted in a significantly smaller diameter of the largest follicle at the end of the pill-free interval and during the first five days of the following treatment cycle. This difference between the two regimens was observed at each interval between treatment cycles during the study.

In this study, it was also important to consider the E2 blood levels. They reflected only the residual follicular activity during the pill-free interval but they also took into account the exogenous E2 due to the study medication during the active treatment sequence. The lower blood E2 found with the 24-day regimen at the end of the second pill-free interval and during the consecutive cycle could also account for the stronger follicular inhibition produced by this regimen.

The gonadotropin profiles explained the stronger suppression of ovarian activity of the 24-day regimen. Increasing the treatment sequence resulted in a delay in the increase in FSH and in significantly lower LH and FSH blood levels at some measurements during the pill-free interval.

Even if there were no significant difference between the two groups, there were in the 24-day regimen group about half fewer women with a follicle larger than 13 mm in diameter, i.e. able to lead to ovulation. Furthermore no follicle reached this value in women who were not completely compliant in the 24-day regimen compared to the 21-day regimen.

The significant difference found between the two regimens in the present study relates to the bleeding profile. The 24-day regimen resulted in a better bleeding pattern: it significantly reduced the total duration of genital bleedings during the study treatment period by approximately 3 days. This reduction was found for both withdrawal and intermenstrual bleedings. The different bleeding patterns could partly explain the significant difference found between the two groups in the change of the red blood cell count and hematocrit during treatment. These parameters slightly decreased with 21-day regimen while they did not change with the 24-day regimen.

Both regimens were similarly potent in inhibiting cervical mucus and in reducing endometrial thickness. Return to ovulation and/or spontaneous menstruation was checked in all women after the end of treatment.

There were no serious adverse events, and no drop-outs for safety reasons. The most frequent adverse events were those usually reported in women treated with hormones.

In conclusion, the monophasic regimen of the subject invention provided a significantly better bleeding pattern when compared with the conventional 21/7 regimen. In addition, the 24-day regimen was associated with a significantly stronger follicular suppression.

### REFERENCES

Astedt, et al. (1977) “The natural oestrogenic hormone oestradiol as a new component of combined oral contraceptives,” Br. Med. J., 1(6056):269.

Bergink, et al. (1981) “Effect of oestriol, oestradiol valerate and ethinyloestradiol on serum proteins in oestrogen-deficient women,” Maturitas, 3(3-4):241-7.

Bonnar, et al. (1987) “Blood coagulation with a combination pill containing gestodene and ethinyl estradiol,” Int. J. Fertil., 32 Suppl:21-8.

Bonnar, J. (1987) “Coagulation effects of oral contraception” Am. J. Obstet. Gynecol. 157: 1042-1048.

Buckman, et al. (1980) “Differential lipemic and proteinemic response to oral ethinyl estradiol and parenteral estradiol cypionate,” Metabolism, 29(9):803-5.

Bazin, et al. (1987) “Effect of nomegestrol acetate, a new 19-nor-progesterone derivative, on pituitary-ovarian function in women,” Br. J. Obstet. Gynaecol., 94(12):1199-204.

Burkman, R. T. (1997) “The estrogen component of OCs: cardiovascular benefits and risks” Int. J. Fertil. Womens Med. Suppl. 1:145-57.

Couzinet B., et al. (1999) “The antigonadotropic activity of a 19-nor-progesterone derivative is exerted both at the hypothalamic and pituitary levels in women,” J. Clin Endocrinol Metab, 84: 4191-4196.

Csemicsky, et al. (1996) “The pharmacodynamic effects of an oral contraceptive containing 3 mg micronized 17 beta-estradiol and 0.150 mg desogestrel for 21 days, followed by 0.030 mg desogestrel only for 7 days,” Contraception, 54(6):333-8.

Daly, L. & Bonnar, J. (1990) “Comparative studies of 30 mu g ethinyl estradiol combined with gestodene and desogestrel on blood coagulation, fibrinolysis, and platelets” Am. J. Obstet. Gynecol. Vol. 163(1).

Fitzgerald, C., et al. (1994) “Comparison of the effects of two monophasic low dose contraceptives on the inhibition of ovulation,” Adv Contracept, 10: 5-18.

Hirvonen et al. (1995) “Oral contraceptive containing natural estradiol for premenopausal women,” Maturitas, 21(1):27-32.

Hirvonen et al. (1988) “New natural oestradiol/cyproterone acetate oral contraceptive for pre-menopausal women,” Maturitas, 10(3):201-13.

Hoffmann, et al. (1998) “Approaches to the replacement of ethinylestradiol by natural 17beta-estradiol in combined oral contraceptives,” Exp. Toxicol. Pathol., 50(4-6):458-64.

Insler, V., et al. (1972) “The cervical score. A simple semiquantitative method for monitoring of the menstrual cycle,” Int. J. Gynaecol. Obst., 10: 223-228.

Lindberg et al. (1989) “A comparison between effects of estradiol valerate and low dose ethinyl estradiol on haemostasis parameters” Thromb Haemost., 61(1):65-9.

Mall-Haefeli, M., et al. (1991) “Clinical experience with Mercilon and Marvelon with special reference to ovarian function,” Geburtshilfe Frauerheilkd, 51:34-38.

Meade, T. W. (1988) “Risks and mechanisms of cardiovascular events in users of oral contraceptives” Am. J. Obstet. Gynecol. 158(6 Pt 2):1646-52.

Neumann (1977) “Pharmacology and potential use of cyproterone acetate,” Horm Metab Res., 9(1):1-13.

O'Brien, P. (1999) “Study confirms tendency towards lower risk of myocardial infarction with second generation oral contraceptives in UK” BMJ., 319(7218):1199.

Odlind, V., et al. (2002) “Can changes in sex hormone binding globulin predict the risk of venous thromboembolism with combined oral contraceptive pills?” Acta Obstet. Gynecol. Scand., 81: 482-490.

Oettel, et al. (1999) “Pharmacokinetics of Dienogest as a Single Drug or in Combination with Estradiol Valerate or Ethinylestradiol,” Med. Actual., 35:27-39.

Paris, et al. (1987) “Extinction of mineralocorticoid effects in 19-norprogesterone derivatives: structure-activity relationships,” J. Pharmacol. Exp. Ther., 243(1):288-91.

Sabra, A. & Bonnar, J. (1983) “Hemostatic system changes induced by 50 g and 30 g estrogen/progestogen oral contraceptives. Modification of estrogen effects by levonorgestrel” J. Reproduc. Med. 28:85-91.

Serup, et al. (1981) “Effectivity and acceptability of oral contraceptives containing natural and artificial estrogens in combination with a gestagen. A controlled double-blind investigation,” Acta Obstet. Gynecol. Scand., 60(2):203-6.

Spitzer (1997) “The 1995 pill scare revisited: anatomy of a non-epidemic” Hum. Reprod. 12(11):2347-57.

Spona, J., et al. (1996) “Shorter pill free interval in combined oral contraceptives decreases follicular development,” Contraception, 54: 71-77.

Sullivan, H., et al. (1999) Effect of 21-day and 24-day oral contraceptive regimens containing gestodene (60 μg) and ethinyl E2 (15 μg) on ovarian activity. Fertil Steril, 72: 115-120.

Van Heusden A. M., Fauser B C J M (1999) “Activity of the pituitary-ovarian axis in the pill-free interval during use of low-dose combined oral contraceptives,” Contraception, 59: 237-243.

Von Schoultz, et al. (1989) “Estrogen therapy and liver function—metabolic effects of oral and parenteral administration,” Prostate., 14(4):389-95.

Wenzl et al (1993) “Ovulation inhibition with a combined oral contraceptive containing 1 mg micronized 17 beta-estradiol,” Fertil. Steril., 60(4):616-9.

World Health Organization Task Force on Oral Contraception (1980) “A randomized, double-blind study of two combined oral contraceptives containing the same progestogen, but different estrogens,” Contraception, 21(5):445-59.

1. A monophasic method of achieving contraception in a human female comprising orally administering to the human female a composition comprising 1.5 mg of 17-beta-estradiol and 2.5 mg of nomegestrol for 24 days followed by a hormone-free period of 4 days.

2. The method of claim 1, wherein the composition is in the form of a tablet.

3. A method of achieving contraception in a human female which comprises repeatedly performing the method of claim 1.

4. The method of claim 3, wherein the repeated performance of the method commences on day 29.

5. The method of claim 1, wherein a placebo is administered daily during the hormone-free period

6. A monophasic method of achieving contraception in a human female wherein the duration of the genital bleeding is reduced, comprising orally administering to the human female a composition comprising 1.5 mg of 17-beta-estradiol and 2.5 mg of nomegestrol acetate for 24 days followed by a hormone-free period of 4 days.

7. The method of claim 6 wherein the composition is in the form of a tablet.

8. A method of achieving contraception in a human female which comprises repeatedly performing the method of claim 6.

9. The method of claim 8, wherein the repeated performance of the method commences on day 29.

10. The method of claim 6, wherein a placebo is administered daily during the hormone-free period.
